SEQUENCE LISTING (8 sequences)

SEQ ID NO: 1 - protein, 605 aa, Homo sapiens
MAGLYSLGVSVFSDQGGRKYMEDVTQIVVEPEPTAEEKPSPRRSLSQPLPPRPSPAALPGGEVSGKGPAVAAREARDPLPDAGASPAPSRCCRRRSSVAFFAVCDGHGGREAAQFAREHLWGFIKKQKGFTSSEPAKVCAAIRKGFLACHLAMWKKLAEWPKTMTGLPSTSGTTASVVIIRGMKMYVAHVGDSGVVLGIQDDPKDDFVRAVEVTQDHKPELPKERERIEGLGGSVMNKSGVNRVVWKRPRLTHNGPVRRSTVIDQIPFLAVARALGDLWSYDFFSGEFVVSPEPDTSVHTLDPQKHKYIILGSDGLWNMIPPQDAISMCQDQEEKKYLMGEHGQSCAKMLVNRALGRWRQRMLRADNTSAIVICISPEVDNQGNFTNEDELYLNLTDSPSYNSQETCVMTPSPCSTPPVKSLEEDPWPRVNSKDHIPALVRSNAFSENFLEVSAEIARENVQGVVIPSKDPEPLEENCAKALTLRIHDSLNNSLPIGLVPTNSTNTVMDQKNLKMSTPGQMKAQEIERTPPTNFKRTLEESNSGPLMKKHRRNGLSRSSGAQPASLPTTSQRKNSVKLTMRRRLRGQKKIGNPLLHQHRKTVCVC

SEQ ID NO: 2 - DNA, 4790 bp, Homo sapiens
ggggaagcgcagtgcgcaggcgcaactgcctggctctgctcgctccggcgctccggcccagctctcgcggacaagtccagacatcgcgcgcccccccttctccgggtccgccccctcccccttctcggcgtcgtcgaagataaacaatagttggccggcgagcgcctagtgtgtctcccgccgccggattcggcgggctgcgtgggaccggcgggatcccggccagccggccatggcggggctgtactcgctgggagtgagcgtcttctccgaccagggcgggaggaagtacatggaggacgttactcaaatcgttgtggagcccgaaccgacggctgaagaaaagccctcgccgcggcggtcgctgtctcagccgttgcctccgcggccgtcgccggccgcccttcccggcggcgaagtctcggggaaaggcccagcggtggcagcccgagaggctcgcgaccctctcccggacgccggggcctcgccggcacctagccgctgctgccgccgccgttcctccgtggcctttttcgccgtgtgcgacgggcacggcgggcgggaggcggcacagtttgcccgggagcacttgtggggtttcatcaagaagcagaagggtttcacctcgtccgagccggctaaggtttgcgctgccatccgcaaaggctttctcgcttgtcaccttgccatgtggaagaaactggcggaatggccaaagactatgacgggtcttcctagcacatcagggacaactgccagtgtggtcatcattcggggcatgaagatgtatgtagctcacgtaggtgactcaggggtggttcttggaattcaggatgacccgaaggatgactttgtcagagctgtggaggtgacacaggaccataagccagaacttcccaaggaaagagaacgaatcgaaggacttggtgggagtgtaatgaacaagtctggggtgaatcgtgtagtttggaaacgacctcgactcactcacaatggacctgttagaaggagcacagttattgaccagattccttttctggcagtagcaagagcacttggtgatttgtggagctatgatttcttcagtggtgaatttgtggtgtcacctgaaccagacacaagtgtccacactcttgaccctcagaagcacaagtatattatattggggagtgatggactttggaatatgattccaccacaagatgccatctcaatgtgccaggaccaagaggagaaaaaatacctgatgggtgagcatggacaatcttgtgccaaaatgcttgtgaatcgagcattgggccgctggaggcagcgtatgctccgagcagataacactagtgccatagtaatctgcatctctccagaagtggacaatcagggaaactttaccaatgaagatgagttatacctgaacctgactgacagcccttcctataatagtcaagaaacctgtgtgatgactccttccccatgttctacaccaccagtcaagtcactggaggaggatccatggccaagggtgaattctaaggaccatatacctgccctggttcgtagcaatgccttctcagagaattttttagaggtttcagctgagatagctcgagagaatgtccaaggtgtagtcataccctcaaaagatccagaaccacttgaagaaaattgcgctaaagccctgactttaaggatacatgattctttgaataatagccttccaattggccttgtgcctactaattcaacaaacactgtcatggaccaaaaaaatttgaagatgtcaactcctggccaaatgaaagcccaagaaattgaaagaacccctccaacaaactttaaaaggacattagaagagtccaattctggccccctgatgaagaagcatagacgaaatggcttaagtcgaagtagtggtgctcagcctgcaagtctccccacaacctcacagcgaaagaactctgttaaactcaccatgcgacgcagacttaggggccagaagaaaattggaaatcctttacttcatcaacacaggaaaactgtttgtgtttgctgaaatgcatctgggaaatgaggtttttccaaacttaggatataagagggctttttaaatttggtgccgatgttgaactttttttaaggggagaaaattaaaagaaatatacagtttgactttttggaattcagcagttttatcctggccttgtacttgcttgtattgtaaatgtggattttgtagatgttagggtataagttgctgtaaaatttgtgtaaatttgtatccacacaaattcagtctctgaatacacagtattcagagtctctgatacacagtaattgtgacaatagggctaaatgtttaaagaaatcaaaagaatctattagattttagaaaaacatttaaactttttaaaatacttattaaaaaatttgtataagccacttgtcttgaaaactgtgcaactttttaaagtaaattattaagcagactggaaaagtgatgtattttcatagtgacctgtgtttcacttaatgtttcttagagccaagtgtcttttaaacattattttttatttctgatttcataattcagaactaaatttttcatagaagtgttgagccatgctacagttagtcttgtcccaattaaaatactatgcagtatctcttacatcagtagcatttttctaaaaccttagtcatcagatatgcttactaaatcttcagcatagaaggaagtgtgtttgcctaaaacaatctaaaacaattcccttctttttcatcccagaccaatggcattattaggtcttaaagtagttactcccttctcgtgtttgcttaaaatatgtgaagttttccttgctatttcaataacagatggtgctgctaattcccaacatttcttaaattattttatatcatacagttttcattgattatatgggtatatattcatctaataaatcagtgaactgttcctcatgttgctgaatttgtagttgttggtttattttaatggtatgtacaagttgagtatcccttatccaaaatgcttgggaccagaagtgtttcagattttttaaaattttggaatatttgctttatactgagcttttgagtgttcccaatctgaaattcaaaatgctctaatgagcatttcctttgagcatcatgcctgctctgaaaaagtttctgattctggagcattttggattttggattttcagattagggatgcttaacctggattaacattctgttgtgccatgatcatgctttacagtgagtgtattttatttatttattattttgtttgtttgtttgagatggagtctcactctgtcatccaggctagagtgcagtggcgtgatctcggctgactgcaacctctgcctcccgggttcaagtgattctcctgcctcaatctctctccccagaagctgggattacaggtgtgtgccaccacacccggctaatttttttttttttttttgagatggagtctagctctgtcatccaggctggagtgcagtggtgtgatctcggctccctgcaacctctgccttctgggttcctgcgattctcctgcctcagcctcctgagtagctgagattacaggcacgcgccactgtgcccagccaatttttgtatttttagtagagatggggtttcacatgtcagtcatgctggtcttgatctcctgacctcgtgatccacccgcctcgacctcccaaagtactgggattacaggcgtgagccaccgcatccggcctgagttttatgctttcaatgtatttcttacatttcagttcaagtgattttcatgtctcagcctcctgagtagctggaactacaggtgcgtgccaccatgcctggctaagttttgtatttttagtagagatgggttttcatcatgttggccaagatggtcttgatctcttgacctcatgatccaccagcctaggcctcccaaagtgctgggattacaggtgtgagccaccgtgcccagccaactatgccattatttaaccatgtccacacattctggttattttcaatattttgcagaagataattcttgatcggtgtgtcttatgccacaaggattaaaatatgtattcattgctacaaaacaatatctcgaaatttagcagtttaaaacaacaaatattatctccagtttctgagcctcagaaatctgagagtggtttagctgggtgatagtctcgtggttttggtcaagctaccaaccagggctacaatctttcgaaggtgtcattggggctagaagatctgcttcccgcaagactcacagctgttggcaggagacctcagtttgttgccacatgttcccctccagagggcctctcacaacatggcagttatttgtccccagagcaagcaacaccggagggcaaggaagaagccatgatgttttttgtaacctagcctctgaaagtgtcataccaattctgtattttgttggtcacacagaccaagtcaactacaacgtgggagactcctacacaaggcatgaattctaggaggtgggcatttttaagtgtcatctggaaggaggctgtcacaacctggaagttaaaagcattgatattctgaaatacagcgtgtataacattgttttagtagggtgtgcaatagttatgttttggtaatagcattaatgaacaatgttattttcatcttccagacatctggaagattgctctagtggagtaaaacatcttaatgtattttgtccctaaataaactatctcactaacaaaaaaaaaaaaaaa

SEQ ID NO: 3 - DNA, 20 bp, Artificial sequence
  Synthetic sequence Forward cDNA-specific primer
accaccagtcaagtcactgg

SEQ ID NO: 4 - DNA, 20 bp, Artificial sequence
  Synthetic sequence Reverse cDNA-specific primer
tctttcgctgtgaggttgtg

SEQ ID NO: 5 - DNA, 23 bp, Artificial sequence
  Synthetic sequence Forward DNA amplification primer
gagagaatgtctaaggtgtagtc

SEQ ID NO: 6 - DNA, 23 bp, Artificial sequence
  Synthetic sequence Reverse DNA amplification primer
gactacaccttagacattctctc

SEQ ID NO: 7 - DNA, 18 bp, Artificial sequence
  Synthetic sequence Forward DNA amplification primer
gatccagaaccattgaag

SEQ ID NO: 8 - DNA, 18 bp, Artificial sequence
  Synthetic sequence Reverse DNA amplification primer
cttcaatggttctggatc

(19) United States
(12) Patent Application Publication
(10) Pub. No.: US 2015/0284806 A1
(43) Pub. Date: Oct. 8, 2015

(54) MATERIALS AND METHODS FOR DETERMINING SUSCEPTIBILITY OR PREDISPOSITION TO CANCER

(71) Applicant: THE INSTITUTE FOR CANCER RESEARCH:ROYAL CANCER HOSPITAL, London (GB)

(72) Inventor: Nazneen Rahman, London (GB)

(21) Appl. No.: 14/646,559

(22) PCT Filed: Nov. 14, 2013
(86) PCT No.: PCT/GB2013/053002

§ 371 Date: May 21, 2015

Related U.S. Application Data
(60) Provisional application No. 61/728,881, filed on Nov. 21, 2012.

(30) Foreign Application Priority Data
Nov. 21, 2012 (GB) 1220924.3

(51) Int. Cl. C12Q 1/68; G06F 19/22; G06F 19/20; G01N 33/574; G06F 19/00

(52) U.S. Cl. CPC C12Q 1/6886; G01N 33/57496; G06F 19/3431; G06F 19/20; G06F 19/22; C12Q 2600/156; G01N 2333/916; G01N 2800/50; C12Q 2600/118

(57) ABSTRACT

Materials and methods for determining the susceptibility or predisposition to cancer are disclosed, and more particularly mutations found in the PPM1D gene that are associated with an increased risk of cancer.

### FIELD OF THE INVENTION

The present invention relates to materials and methods for determining the susceptibility or predisposition to cancer and more particularly mutations found in the PPM1D gene that are associated with an increased risk of cancer.

### BACKGROUND OF THE INVENTION

Rare genetic variation is thought to be a key determinant of genetic predisposition to breast and ovarian cancers. Linkage analysis and cloning studies have implicated rare mutations in the DNA repair genes BRCA1 and BRCA2 as high-penetrance determinants of breast and ovarian cancer susceptibility. More recently, case-control studies have linked loss of function (LOF) and often protein truncating mutations in other genes with roles in DNA repair such as PALB2, ATM, CHEK2, BRIP1, RAD51C and RAD51D in breast and/or ovarian cancer risk. However, the majority of familial risk to these cancers remains unexplained.

### SUMMARY OF THE INVENTION

Broadly, the present invention is based on research to identify additional genes associated with cancer predisposition, especially breast and ovarian cancer predisposition. This involved screening lymphocyte DNA for mutations in 507 genes encoding proteins implicated in DNA repair in pooled samples from 1,150 individuals with breast cancer, 69 of whom also had ovarian cancer. Of the 34,564 variants called, 1,044 were identified as protein truncating variants (PTVs). Because of the strong association of this class of mutation with breast and ovarian cancer predisposition, genes were stratified by the number of PTVs and PPM1D was identified as the gene with the strongest signal in this analysis (excluding the known predisposition genes).

PPM1D (protein phosphatase, Mg²⁺/Mn²⁺ dependent 1D; also known as WIP1) encodes a 605 amino acid protein with an N-terminal phosphatase catalytic domain and a C-terminal domain that contains a putative nuclear localisation signal (FIG. 2b and FIG. 2c). PPM1D has been shown to be involved in the negative regulation of several tumour suppressor pathways. PPM1D expression is upregulated in response to DNA damage through TP53/p53, and functions to dephosphorylate and downregulate the activity of MAPK/p38, thereby suppressing the activation of proteins associated with ATM/ATR-initiated DNA damage response (DRR), including tumour suppressors such as p53, ATM and CHK2. Thus it has been proposed that a primary role of PPM1D is as a homeostatic regulator of the DDR, facilitating return of cells to their normal state after repair of damaged DNA. Moreover, PPM1D has been shown to be amplified and overexpressed in multiple human tumours, including breast cancers and ovarian clear cell carcinoma.

Sanger sequencing was subsequently performed on 13,642 individuals (7,781 individuals with breast and or ovarian cancer and 5,861 population controls) to further explore the role of PPM1D in cancer susceptibility. This identified a total of 25 PTVs clustered in the final exon of PPM1D in individuals with breast and/or ovarian cancer (18 in 6,912 individuals with breast cancer, 12 in 1,121 individuals with ovarian cancer) and 1 in controls (Table 1, FIG. 1, Table 3).

Retrospective cohort analysis demonstrated that PPM1D PTV carriers had a relative risk of breast cancer of 2.7 (95% CI: 1.3-5.3; P=5.38×10⁻³), which translates to approximately 23% cumulative risk by age 80, and a relative risk of ovarian cancer of 11.5 (95% CI: 4.3-30.4; P=9.95×10⁻⁷), which translates to approximately 18% cumulative risk by age 80.

Thus, the present invention represents the first evidence that mutations in the PPM1D gene, especially protein truncating mutations, are linked to predisposition to cancer, and in particular breast and ovarian cancer.

Moreover, the frequency of PPM1D PTVs was significantly higher in BRCA1/2 mutation carriers with breast and/or ovarian cancer compared to population controls (4/773 vs. 1/5861; P=8.30×10⁻⁴), suggesting that PPM1D PTVs are associated with an increased risk of cancer in BRCA1/2 mutation carriers.

Thus, the present invention provides evidence for an interaction between PPM1D PTV mutations and previously identified risk alleles in predisposition to cancer, especially breast and ovarian cancer.

Sequencing chromatograms showed unusually low signal for the PTVs, suggesting that rather than being heterozygous, the mutations were mosaic in the lymphocyte DNA (FIG. 2a). PTV mutations were confirmed to be mosaic by deep PCR amplicon sequencing (FIG. 2b, Table 3), multiplex ligation-dependent probe amplification (MLPA; FIG. 4), re-sequencing of the DNA repair panel in six cases individually (Table 3) and family studies which showed none of 14 relatives carried the PPM1D mutation identified in the proband (FIG. 2c).

Sanger sequencing and MPLA analysis were unable to identify PPM1D mutations in any of eight tumours from five PPM1D PTV carriers, suggesting the mechanism underlying association for PPM1D PTV mutations differs from that of other cancer-associated DNA repair genes.

The PPM1D PTVs identified were downstream of the phosphatase catalytic domain but upstream or disruptive of the nuclear localisation signal (FIG. 1). Functional studies showing that p53 suppression is enhanced in cells transfected with cDNA expression constructs for two of the PTV mutations (PPM1D c.1384C>T; case 6 and PPM1D c.1420delC; case 7) relative to cells transfected with a wildtype PPM1D cDNA construct suggested the PTVs result in the production of a hyperactive PPM1D isoform (FIG. 3).

In a first aspect, the present invention provides a method for determining whether an individual has an increased susceptibility to cancer, the method comprising determining in a sample obtained from the individual the presence of a mutation in the PPM1D gene, or a polypeptide encoded by the PPM1D gene wherein the presence of a mutation is indicative of increased risk of cancer. In a preferred embodiment, the cancer is breast or ovarian cancer. In a further preferred embodiment, the mutations are mutations leading to increased PPM1D activity. In a further preferred embodiment, the mutations are truncating mutations.

Examples of truncating mutations are disclosed in Table 1.

Additional mutations in the PPM1D gene that may be used in the present invention include any other mutation in the PPM1D gene, or any other mutation encoding a truncated PPM1D polypeptide.

In a further aspect, the present invention provides a method which comprises having determined whether an individual has an increased susceptibility or predisposition to cancer according to the method of any one of the preceding claims, one or more of the further step of:
(a) correlating the presence of said mutations to a susceptibility or predisposition to breast cancer or ovarian cancer; and/or
(b) saving data representing the result of the test on a recordable media; and/or
(c) transmitting the data representing the result of the test to a recipient.

In a further aspect, the present invention provides a kit for detecting mutations in the PPM1D gene associated with a susceptibility to cancer according to any one of the preceding claims, the kit comprising:
(a) one or more sequence specific probes as disclosed herein; and/or
(b) one or more sequence specific primers for amplifying a portion of the PPM1D nucleic acid sequence as disclosed herein; and/or
(c) one or more specific binding partners capable of specifically binding to full length or truncated PPM1D polypeptide as disclosed herein; and/or
(d) a microarray as disclosed herein.

In a further aspect, the present invention provides novel nucleic acid and polypeptide sequences that includes an isolated nucleic acid molecule encoding the PPM1D gene having at least 90% nucleic acid sequence identity with the sequence as set out in SEQ ID NO: 2, wherein the nucleic acid comprises one of the mutations set out in Table 1 or a further mutation as disclosed above.

In further aspects, the present invention further relates to a replicable vector comprising these nucleic acid sequences and to host cells transformed with the vector, e.g. for use in expressing PPM1D nucleic acid by culturing the host cells so that the polypeptide encoded by the PPM1D nucleic acid is produced. The present invention also provides polypeptides encoded by these nucleic acid molecules and antibodies capable of specifically binding to the PPM1D polypeptides.

In further aspects, the present invention further relates to the use of inhibitors and pharmaceutical compositions comprising inhibitors of PPM1D for use in a method of treating cancer, wherein the method comprises determining whether an individual has an increased predisposition to cancer and treating the individual with the PPM1D inhibitor.

Embodiments of the present invention will now be described by way of example and not limitation with reference to the accompanying figures. However various further aspects and embodiments of the present invention will be apparent to those skilled in the art in view of the present disclosure. “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described

### BRIEF DESCRIPTION OF THE FIGURES AND SEQUENCES

FIG. 1. Clustering of cancer predisposing mutations in PPM1D. a, PPM1D gene with region targeted by mutations (mutation cluster region) in blue; b, PPM1D protein showing position of mutation cluster region downstream of the phosphatase domain and upstream/overlapping the nuclear localisation signal (NLS); c, mutation cluster region showing position of mutations. The numbers above give the position of the mutations and correspond to the IDs in Table 1.

FIG. 2. PPM1D mutations are mosaic in lymphocyte DNA. a, Sanger sequencing traces showing mutant allele is lower in genomic DNA extracted from peripheral blood lymphocytes (gDNA) than typical for heterozygous mutations. The cDNA analysis demonstrates that the mutations lead to a truncated product rather than nmRNA decay. b, deep PCR amplicon sequencing showing heterozygous BRCA1/2 variants at 50% (open dots) whereas the PPM1D mutation is present at a lower percentage (red dots). c, Haplotype analysis in two families. The offspring of PPM1D mutation carriers have different maternal haplotypes spanning the PPM1D locus (highlighted), but neither carry the mutation, indicating that it is either not present, or mosaic in the germline of the proband.

FIG. 3. The effect of mutant PPM1D isoforms on p53 activation. p53 wildtype U2OS human osteosarcoma cells were transfected with PPM1D cDNA expression constructs and exposed to ionising irradiation (5 Grays). At 30 minute and four hour intervals after IR exposure whole cell lysates were generated and western blotted to estimate the IR induced activation of p53. Western blots showing p53 and actin (loading control) protein levels at different times (in hours) after IR exposure are shown. ‘Empty’ represents cells transfected with an empty expression construct, ‘PPM1D WT’ represents cells transfected with a wildtype PPM1D cDNA expression construct and ‘PPM1D c.1384C>T’ and ‘PPM1D c.1420delC’ represent cells transfected with mutant PPM1D cDNA constructs. The suppression of p53 was enhanced in cells transfected with the mutant constructs suggesting these alleles encode hyperactive PPM1D isoforms.

FIG. 4. MLPA profiles showing PPM1D mutations.

SEQ ID NO: 1 shows the amino acid sequence of PPM1D.

SEQ ID NO: 2 shows the nucleic acid coding sequence of the PPM1D gene.

### DETAILED DESCRIPTION

### PPM1D Gene and Polypeptide Sequences

The PPM1D gene and polypeptide sequences are disclosed in Ali, A. Y. et al., Oncogene, 31(17), 2175-2186 (2012) and are publicly available on GenBank as sequence accession numbers NM_{—}003620 and NP_{—}003611. The polypeptide sequence is 605 amino acids in length and is provided a SEQ ID NO: 1. The coding sequence of the PPM1D gene is reproduced herein as SEQ ID NO: 2. PPM1D nucleic acid includes the sequence shown in SEQ ID NO: 2, alleles and sequence variants thereof and complementary sequences of any of these nucleic acids. The numbering used herein refers to these sequences and in particular in Table 1 to the coding sequence of the PPM1D gene shown in SEQ ID NO: 2. However, the present invention is also applicable to the use of alleles and sequence variants of this gene that may include one or more of the mutations as disclosed herein.

PPM1D nucleic acid and amino acid sequences preferably have at least 90% sequence identity, more preferably 98% sequence identity, and most preferably at least 98% sequence identity, to their respective sequences set out in SEQ ID NO: 1 and 2. “Percent (%) amino acid sequence identity” with respect to the PPM1D polypeptide sequences identified herein is defined as the percentage of amino acid residues in a candidate sequence that are identical with the amino acid residues in the PPM1D sequence, after aligning the sequences and introducing gaps, if necessary, to achieve the maximum percent sequence identity, and not considering any conservative substitutions as part of the sequence identity. The % identity values can be generated by WU-BLAST-2 which was obtained from [Altschul et al, Methods in Enzymology, 266:460-480 (1996); http://blast.wustl/edu/blast/README.html]. WU-BLAST-2 uses several search parameters, most of which are set to the default values. The adjustable parameters are set with the following values: overlap span=1, overlap fraction=0.125, word threshold (T)=11. The HSPS and HSPS2 parameters are dynamic values and are established by the program itself depending upon the composition of the particular sequence and composition of the particular database against which the sequence of interest is being searched; however, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

Similarly, “percent (%) nucleic acid sequence identity” with respect to the coding sequence of the PPM1D polypeptides identified herein is defined as the percentage of nucleotide residues in a candidate sequence that are identical with the nucleotide residues in the PPM1D coding sequence as provided in SEQ ID NO: 2. The identity values used herein were generated by the BLASTN module of WU BLAST-2 set to the default parameters, with overlap span and overlap fraction set to 1 and 0.125, respectively.

Particular mutant alleles of the present invention are set out in Table 1 and are described using the nomenclature in Nomenclature for the description of human sequence variations, den Dunnen, J T and Antonarakis, S E, Hum. Genet., July; 109(1):121-4, 2001. These mutations are generally associated with the production of truncated forms of PPM1D polypeptide shown in the experimental work described herein to be associated with susceptibility to cancer, and especially to breast cancer or ovarian cancer. Implications for screening, e.g. for diagnostic or prognostic purposes, are discussed below.

The finding of mutations to the wild type PPM1D gene sequence means that, in some aspects, the present invention provides novel PPM1D nucleic acid sequences, in particular the mutations set out in Table 1 or described elsewhere in the present application. Generally, nucleic acid according to the present invention is provided as an isolate, in isolated and/or purified form, or free or substantially free of material with which it is naturally associated, such as free or substantially free of nucleic acid flanking the gene in the human genome, except possibly one or more regulatory sequence(s) for expression. Nucleic acid may be wholly or partially synthetic and may include genomic DNA, cDNA or RNA. Where nucleic acid according to the invention includes RNA, reference to the sequence shown should be construed as reference to the RNA equivalent, with U substituted for T.

Nucleic acid sequences encoding all or part of the PPM1D gene and/or its regulatory elements can be readily prepared by the skilled person using the information and references contained herein and techniques known in the art (for example, see Sambrook, Fritsch and Maniatis, “Molecular Cloning, A Laboratory Manual, Cold Spring Harbor Laboratory Press, 1989, and Ausubel et al., Short Protocols in Molecular Biology, John Wiley and Sons, 1992). These techniques include (i) the use of the polymerase chain reaction (PCR) to amplify samples of such nucleic acid, e.g. from genomic sources, (ii) chemical synthesis, or (iii) preparing cDNA sequences.

In order to obtain expression of the PPM1D nucleic acid sequences, including the novel mutated sequences disclosed herein, the sequences can be incorporated in a vector having control sequences operably linked to the PPM1D nucleic acid to control its expression. The vectors may include other sequences such as promoters or enhancers to drive the expression of the inserted nucleic acid, nucleic acid sequences so that the PPM1D polypeptide is produced as a fusion and/or nucleic acid encoding secretion signals so that the polypeptide produced in the host cell is secreted from the cell. PPM1D polypeptide can then be obtained by transforming the vectors into host cells in which the vector is functional, culturing the host cells so that the PPM1D polypeptide is produced and recovering the PPM1D polypeptide from the host cells or the surrounding medium. Prokaryotic and eukaryotic cells are used for this purpose in the art, including strains of E. coli, yeast, and eukaryotic cells such as COS or CHO cells. The choice of host cell can be used to control the properties of the PPM1D polypeptide expressed in those cells, e.g. controlling where the polypeptide is deposited in the host cells or affecting properties such as its glycosylation.

### Methods of Determining the Presence of Mutations

A wide range of techniques are known in the art for determining the presence of a presence of mutations in a gene such as PPM1D, or in the polypeptide encoded by it. These techniques may be employed by the skilled person for use in accordance with the present invention. In general, the purpose of carrying of the methods disclosed herein on a sample from an individual is to determine whether the individual carries a PPM1D mutation and is at increased risk of developing cancer. The purpose of such analysis may be used for diagnosis or prognosis, e.g. to serve to detect the presence of an existing cancer, to help identify the type of cancer, to assist a physician in determining the severity or likely course of the cancer and/or to optimise treatment of it. Additionally, the methods can be used to detect PPM1D mutations that are statistically associated with a susceptibility to cancer in the future, e.g. breast cancer or ovarian cancer, identifying individuals who would benefit from regular screening to provide early diagnosis of cancer or from risk-reducing strategies, such as preventative surgery, or for whom changes in lifestyle or diet may help to ameliorate the increased susceptibility to a particular form of cancer.

Broadly, the methods divide into those screening for the presence of PPM1D nucleic acid sequences and those that rely on detecting the presence of PPM1D polypeptide. Exemplary techniques and their advantages and disadvantages are reviewed in Nature Biotechnology, 15:422-426, 1997. The methods make use of biological samples from individuals that may contain the nucleic acid or polypeptides. Examples of biological samples include blood (including cells isolated from blood, such as lymphocytes), plasma, serum, saliva and tissue samples (including biopsies).

Nucleic acid based testing may be carried out using preparations containing genomic DNA, cDNA and/or mRNA. Testing cDNA or mRNA has the advantage of the complexity of the nucleic acid being reduced by the absence of intron sequences, but the possible disadvantage of extra time and effort being required in making the preparations. RNA is more difficult to manipulate than DNA because of the wide-spread occurrence of RNases.

Techniques that involve looking for mutations in PPM1D nucleic acid sequence include direct sequencing, restriction fragment length polymorphism (RFLP) analysis, single-stranded conformation polymorphism (SSCP), heteroduplex analysis, PCR amplification of specific alleles, amplification of DNA target by PCR followed by a mini-sequencing assay, allelic discrimination during PCR, Genetic Bit Analysis, pyrosequencing, oligonucleotide ligation assay, or analysis of melting curves.

Techniques that involve looking for mutations in PPM1D polypeptides include the use of specific binding members such as antibodies to detect mutated and/or normal PPM1D polypeptides.

### Restriction Digest

The presence of differences in sequence of nucleic acid molecules may be detected by means of restriction enzyme digestion, such as in a method of DNA fingerprinting where the restriction pattern produced when one or more restriction enzymes are used to cut a sample of nucleic acid is compared with the pattern obtained when a sample containing the normal gene or a variant or allele is digested with the same enzyme or enzymes.

### Probes

Mutations in nucleic acid may also be screened using a mutant- or allele-specific probe. Such a probe corresponds in sequence to a region of the PPM1D gene, or its complement, containing a sequence mutation known to be associated with cancer susceptibility, for example as set out in Table 1. Under suitably stringent conditions, specific hybridisation of such a probe to test nucleic acid is indicative of the presence of the sequence alteration in the test nucleic acid. For efficient screening purposes, more than one probe may be used on the same test sample. This approach may be adapted to use a microarray as discussed in more detail below.

The binding of the probe to target nucleic acid (e.g. DNA) may be measured using any of a variety of techniques at the disposal of those skilled in the art. For instance, probes may be radioactively, fluorescently or enzymatically labelled. Other methods not employing labelling of probe include examination of restriction fragment length polymorphisms, amplification using PCR, RNase cleavage and allele specific oligonucleotide probing.

Probing may employ the standard Southern blotting technique. For instance DNA may be extracted from cells and digested with different restriction enzymes. Restriction fragments may then be separated by electrophoresis on an agarose gel, before denaturation and transfer to a nitrocellulose filter. Labelled probe may be hybridised to the DNA fragments on the filter and binding determined. DNA for probing may be prepared from RNA preparations from cells.

Those skilled in the art are well able to employ suitable conditions of the desired stringency for selective hybridisation, taking into account factors such as the length of the probe and base composition, temperature and so on. By way of example, stringent conditions include those that: (1) employ low ionic strength and high temperature for washing, for example 0.015 M sodium chloride/0.0015 M sodium citrate/O.1% sodium dodecyl sulfate at 50° C.; (2) employ during hybridisation a denaturing agent, such as formamide, for example, 50% (v/v) formamide with 0.1% bovine serum albumin/0.l % Ficoll/0.1% polyvinylpyrrolidone/50 mM sodium phosphate buffer at pH 6.5 with 760 mM sodium chloride, 75 mM sodium citrate at 42° C.; or (3) employ 50% formamide, 5×SSC (0.75 M NaCl, 0.075 M sodium citrate), 50 mM sodium phosphate (pH 6 8), 0.1% sodium pyrophosphate, 5×Denhardt's solution, sonicated salmon sperm DNA (50 mg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

Approaches which rely on hybridisation between a probe and test nucleic acid and subsequent detection of a mismatch may be employed. Under appropriate conditions (temperature, pH etc.), an oligonucleotide probe will hybridise with a sequence which is not entirely complementary. The conditions of the hybridisation can be controlled to minimise non-specific binding, and preferably stringent to moderately stringent hybridisation conditions are preferred. The skilled person is readily able to design such probes, label them and devise suitable conditions for the hybridisation reactions, assisted by textbooks such as Sambrook et al (1989) and Ausubel et al (1992). The degree of base-pairing between the two molecules will be sufficient for them to anneal despite a mismatch. Various approaches are well known in the art for detecting the presence of a mismatch between two annealing nucleic acid molecules.

For instance, RNase A cleaves at the site of a mis-match. Cleavage can be detected by electrophoresing test nucleic acid to which the relevant probe or probe has annealed and looking for smaller molecules (i.e. molecules with higher electrophoretic mobility) than the full length probe/test hybrid. Other approaches rely on the use of enzymes such as resolvases or endonucleases.

Thus, an oligonucleotide probe that has the sequence of a region of the normal PPM1D gene (either sense or anti-sense strand) in which mutations associated with cancer susceptibility are known to occur (e.g. see Table 1) may be annealed to test nucleic acid and the presence or absence of a mismatch determined. Detection of the presence of a mismatch may indicate the presence in the test nucleic acid of a mutation associated with cancer susceptibility. On the other hand, an oligonucleotide probe that has the sequence of a region of the PPM1D gene including a mutation associated with cancer susceptibility may be annealed to test nucleic acid and the presence or absence of a mismatch determined. The absence of a mismatch may indicate that the nucleic acid in the test sample has the normal sequence. In either case, a plurality of probes to different regions of the gene may be employed.

### PCR Methods

Allele or variant-specific oligonucleotides may similarly be used in PCR to specifically amplify particular sequences if present in a test sample. Assessment of whether a PCR band contains a gene variant may be carried out in a number of ways familiar to those skilled in the art. The PCR product may for instance be treated in a way that enables one to display the mutation or polymorphism on a denaturing polyacrylamide DNA sequencing gel, with specific bands that are linked to the gene variants being selected. PCR techniques for the amplification of nucleic acid are described in U.S. Pat. No. 4,683,195, Mullis et al., Cold Spring Harbor Symp. Quant. Biol., 51:263, (1987), Ehrlich (ed), PCR technology, Stockton Press, NY, 1989, Ehrlich et al., Science, 252:1643-1650, (1991), “PCR protocols; A Guide to Methods and Applications”, Eds. Innis et al., Academic Press, New York, (1990).

Multiplex PCR can be used to determine the presence of mutations in a gene such as PPM1D. Multiple primer pairs that produce amplicons of varying sizes are used in a single PCR reaction which are then visualised as above. Alternatively, products can be sequenced using one of the methods described below, as for example in deep PCR amplicon sequencing. Multiplex ligation-dependent probe amplification (MPLA) is a variation of multiplex PCR in which a single primer pair amplifies multiple targets, and can be used to discriminate sequences with single nucleotide resolution.

### Heteroduplex Analysis

Mutations in a gene such as PPM1D may be detected by heteroduplex analysis of a PCR-amplified target. Control and sample PCR products are mixed, denatured and allowed to anneal, and the products are resolved by electrophoresis. Mismatches between control and sample sequences will result in the formation of a heteroduplex, with a perturbed structure compared to that of the homoduplex, retarding mobility during electrophoresis. Appropriate temperatures for denaturation and annealing, and electrophoresis conditions for heteroduplex analyses are well known to those skilled in the art.

### Melting Curve Analysis

A gene or region of interest within a gene such as PPM1D may be PCR-amplified and analysed for mutations based on melting curve analysis. The temperature-dependent dissociation of the DNA strands can be measured by, for example, UV absorbance and fluorescence from DNA intercalating fluorophores or labelled probes. Dissociation is sequence-specific and so mutations may be identified as departures from the trajectory of absorbance/fluorescence vs. temperature relationship from a reference sequence, such as SEQ ID NO: 2.

### Sequencing

Nucleic acid in a test sample may be sequenced and the sequence compared with the sequence shown in SEQ ID NO: 2, for example to determine whether the sequence contains a truncating mutation, such as one of the mutations shown in Table 1, and hence is associated with a susceptibility to cancer. Since it will not generally be time or labour efficient to sequence all nucleic acid in a test sample, or even the whole PPM1D gene, a specific amplification reaction such as PCR using one or more pairs of primers may be employed to amplify the region of interest in the nucleic acid, for instance the PPM1D gene or a particular region in which mutations associated with cancer susceptibility occur. Exemplary primers for this purpose can be designed by the skilled person based on the information provided herein. The amplified nucleic acid may then be sequenced as above and/or tested in any other way to determine the presence or absence of a particular feature. Nucleic acid for testing may be prepared from nucleic acid removed from cells or in a library using a variety of other techniques such as restriction enzyme digest and electrophoresis. The sequence of an RNA molecule may be determined by first synthesising cDNA through means well known in the art, which is subsequently sequenced.

Sequencing may be performed using the classic chain termination method, or one of several high-throughput, next generation sequencing (NGS) methodologies, reviewed by Metzker, M. L., Nat Rev Genet 2010 January; 11(1): 31-46. These techniques have in common that they allow time- and cost-effective reconstruction of a DNA sequence by sequencing short, overlapping portions of a fragmented DNA sample in parallel, which are subsequently aligned to reference sequences. Illumina sequencing, 454 pyrosequencing, Heliscope single molecule sequencing, single molecule real time (SMRT) sequencing and Ion semiconductor sequencing platforms are based on the “sequencing by synthesis” principle, determining the sequence of a template strand of DNA through the detection of signals emitted as bases are incorporated into a newly-synthesised complementary strand. Polony sequencing, SOLiD sequencing and DNA nanoball sequencing platforms are based on the “sequencing by ligation” principle, which detect signals emitted from labelled nucleotides as they are ligated by DNA ligase, following recognition of complementary nucleotides in the strand to be sequenced.

Further sequencing technologies are under development and may be employed to determine the presence of a mutation in a gene such as PPM1D.

A gene such as PPM1D may be sequenced as part of whole genome sequencing or exome (i.e. the coding regions of the genome) sequencing projects, or as a member of a panel of disease-associated candidate genes in a targeted sequencing approach. An example of such a targeted disease-associated candidate gene sequencing panel is the Illumina TruSight Cancer panel.

NGS methodologies may be employed on multiple, pooled samples (for example, from individuals with a certain disease or prognosis) that have been enriched using labelled probes for a region or regions of interest (such as PPM1D) to effectively catalogue sequence variation. Sequencing results can be compared with those from samples from other groups (for example, healthy control individuals or those with a different disease phenotype) to implicate certain variants as determinants of disease susceptibility or prognosis.

Moreover, the described sequencing methodologies may be used in independently of one another on the same sample to facilitate the identification of rare and/or mosaic genetic mutations. The combined use of techniques has the advantage of increased power over methods used in isolation, with improved coverage (sequence reads per nucleotide position) of the region of interest.

### Informatics

The proliferation of high-throughput technologies for the analysis of nucleic acids has necessitated the development of informatics tools for the appropriate management and interpretation of data. Accordingly, the present invention provides means for analysing results generated by the above described technologies, wherein the means are the application of a statistical algorithm and/or computer programme to map sequence reads to the gene SEQ ID NO: 2 and polypeptide SEQ ID NO: 1 and identify departures from said sequences. Informatics tools may also be employed to assist the interpretation of sequencing data. F or example, identified mutations may be grouped by type, location, frequency or predicted effect and inform study design for downstream functional analysis. Examples of such statistical algorithms are Stampy (Genome Res, 21(6):936-939, 2011), BWA (Bioinformatics, 25(14):1754-1760, 2009), SOAP2 (Bioinformatics, 25(15):1966-1967, 2009) and Bowtie (Genome Biol, 10(3):R25, 2009). Examples of such computer programmes are Platypus (www.well.ox.ac.uk/platypus), Mutation Surveyor and Genemarker (both SoftGenetics).

### Microarrays

There is an increasing tendency in the diagnostic field towards miniaturisation of assays, e.g. making use of binding agents (such as antibodies or nucleic acid sequences) immobilised in small, discrete locations as arrays on solid supports or on diagnostic chips. The use of microarrays can be particularly valuable as they can provide great sensitivity, particularly through the use of fluorescent labelled reagents, require only very small amounts of biological sample from individuals being tested and allow a variety of separate assays can be carried out simultaneously. This latter advantage can be useful as it provides an assay for different mutations in the PPM1D gene or mutations in other genes to be carried out using a single sample, e.g. in forms of genetic profiling.

Microarrays are libraries of biological or chemical entities immobilised in a grid/array on a solid surface and methods for making and using microarrays are well known in the art. A variation on this theme is immobilisation of these entities onto beads, which are then formed into a grid/array. The entities immobilised in the array can be referred to as probes. These probes interact with targets (a gene, mRNA, cDNA, protein, etc.) and the extent of interaction is assessed using fluorescent labels, colorimetric/chromogenic labels, radioisotope labels or label-free methods (e.g. scanning Kelvin microscopy, mass spectrometry, surface plasmon resonance, etc.). The interaction may include binding, hybridization, absorption or adsorption. The microarray process provides a combinatorial approach to assessing interactions between probes and targets. The basic nucleic acid microarray concept is described in U.S. Pat. Nos. 5,700,637 and 6,054,270.

One type of array uses nucleic acid molecules as the probes. A DNA microarray is a collection of microscopic DNA spots attached to a solid substrate, e.g. glass, plastic or silicon chip, forming an array. DNA microarrays are now commercially available. There are three basic forms: spotted microarrays, lithographic microarrays and bead-based systems. Each involves analysing DNA sequences by the immobilisation of cDNA probes or in situ creation of oligonucleotide sequences and subsequent hybridisation with target mRNA/cDNA complementary to the probes. Often the target cDNA are fluorescently labelled. Sequencing by hybridization approaches are described, for example, in U.S. Pat. Nos. 6,913,879, 6,025,136, 6,018,041, 5,525,464 and 5,202,231.

Two approaches exist to the creation and immobilisation of DNA probes. In the first approach oligonucleotide sequences are built in situ base by base on the chip. In the second, cDNA or oligonucleotide probes are deposited on the array using contact or non-contact printing methods.

In the spotted microarray approach, oligonucleotides, cDNA or small fragments of PCR products corresponding to mRNAs are printed in an array pattern on a solid substrate by either a spotting robot using pins or variations on ink-jet printing methods. The spots are typically in the 30-500 mm size range with separations of the order of 100 mm or more. A lack of uniformity of spot size, variations of spot shape and donut or ring-stain patterns caused during the drying of spots can result in non-uniform immobilisation of the DNA and hence non-uniform fluorescence following the hybridisation.

In lithographic microarrays, sequences of oligonucleotides (A, C, T, G) are built up by selective protection and deprotection of localised areas of the substrate. This approach has been employed, inter alia, by Affymetrix. Affymetrix chips generally provide higher probe densities (spot sizes of the order of 10 mm or greater), but have shorter sequence lengths than in spotted or bead microarrays. The fluorescent labelling of target cDNA remains a key part of the detection strategy.

The photolithographic approach is described in U.S. Pat. Nos. 6,045,996 and 5,143,854.

An alternative method for making arrays employs bead based microarrays. An example of this approach is the system used by Illumina (http://www.illumina.com/) in which probes are immobilised on small (3-5 μm diameter) beads. After hybridisation the beads are cast onto a surface and drawn into wells by surface tension. In the Illumina system, the wells are etched into the ends of optical fibres in fibre bundles. The fluorescence signal is then read for each bead. The method includes a tagging of each bead so that the bioactive agent on each bead can be decoded from the probe position and a decoding system is needed to distinguish the different probes used. The bead based system is described in U.S. Pat. Nos. 6,023,540, 6,327,410, 6,266,459, 6,620,584 and 7,033,754.

Thus, in one embodiment, the present invention provides means for the detection of any departure from the sequence of SEQ ID NO: 2.

### Antibodies

There are various methods for determining the presence or absence in a test sample of a mutated form of the PPM1D polypeptide. For example, a sample may be tested for the presence of a binding partner for a specific binding member such as an antibody (or mixture of antibodies), specific for one or more particular variants of the polypeptide, for example the normal PPM1D polypeptide and mutated forms thereof.

In such cases, the sample may be tested by being contacted with a specific binding member such as an antibody under appropriate conditions for specific binding, before binding is determined, for instance using a reporter system as discussed. Where a panel of antibodies is used, different reporting labels may be employed for each antibody so that binding of each can be determined.

A specific binding member such as an antibody may be used to isolate and/or purify its binding partner polypeptide from a test sample in preference to other components that may be present in the sample. This may be used to determine whether the polypeptide has the sequence shown in SEQ ID NO: 1, or if it is a mutant form. Amino acid sequence is routine in the art using automated sequencing machines. A “specific binding pair” comprises a specific binding member (sbm) and a binding partner (bp) which have a particular specificity for each other and which in normal conditions bind to each other in preference to other molecules. The skilled person will be able to think of many other examples and they do not need to be listed here. It has become a matter of routine in the art for the skilled person to make antibodies that are capable of specifically binding to different polypeptides.

The reactivities of antibodies on a sample may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule.

One favoured mode is by covalent linkage of each antibody with an individual fluorochrome, phosphor or laser dye with spectrally isolated absorption or emission characteristics. Suitable fluorochromes include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed.

As with the above described DNA-based microarrays, the same principles have been extended to protein and chemical microarrays. In these cases the probes immobilised on the surface are specific proteins, antibodies, small molecule compounds, peptides, carbohydrates, etc. rather than DNA sequences. The targets are complex analytes, such as serum, total cell extracts, and whole blood. The key concepts of an array of probes, which undergo selective binding/interaction with a target and which are then interrogated via, for example, a fluorescent, colorimetric or chemiluminescent signal remain central to the method.

A review of ideas on protein and chemical microarrays is given by Xu and Lam in “Protein and Chemical Microarrays—Powerful Tools for Proteomics”, J Biomed., 2003(5): 257-266, 2003. This reference also provides the historical sequence in the development of DNA microarrays. Current research is also extending the microarray concept to include microarrays of cells. A review of patent issues related to early microarrays is given Rouse and Hardiman (“Microarray technology—an intellectual property retrospective”, Pharmacogenomics, 4(5): 623-632, 2003).

Accordingly, in a further aspect, the present invention provides a microarray, or the components for forming a microarray (e.g. a bead array), wherein the microarray comprises one or more binding agents present or locatable on a substrate at a plurality of locations, wherein the one or more binding agents are capable of specifically binding to PPM1D nucleic acid containing a truncating mutation or to a truncated PPM1D polypeptide encoded by the nucleic acid. The microarray will preferably also comprise a plurality of further binding agents for carrying out other tests on the sample, for example to determine the presence of other mutations that are associated with a susceptibility to a disease or condition, such as cancer.

### Kits

In a further aspect, the present invention provides kits for carrying out the methods disclosed herein. The components of the kit will be dependent on whether the method is for determining the presence of a mutation in the PPM1D gene, or a polypeptide encoded by the PPM1D gene, for example the presence of a truncating mutation, or truncated polypeptide.

Generally, the components of the kit will be provided in a suitable form or package to protect the contents from the external environment. The kit may also include instructions for its use and to assist in the interpretation of the results of the test. The kit may also comprise sampling means for use in obtaining a test sample from an individual, e.g. a swab for removing cells from the buccal cavity or a syringe for removing a blood sample (such components generally being sterile).

In one embodiment, the kit may comprise a microarray as described above, optionally in combination with other reagents, such as labelled developing reagents, useful for carrying out testing with the assay. The microarray is preferably a nucleic acid array.

In other embodiments, the kit may be for use in PCR based testing according to the methods disclosed herein and accordingly may comprise one or more primers suitable for amplifying a portion of the PPM1D nucleic acid sequence where one of the mutations associated with a susceptibility to cancer are located. The kit may include instructions for use of the nucleic acid, e.g. in PCR and/or a method for determining the presence of nucleic acid of interest in a test sample. In addition to one or more primers (or pairs of primers), the kit may also one or more further reagents required for the reaction, such as polymerase, nucleosides, buffer solution etc. The nucleic acid primer may also be labelled, for example to facilitate detection and/or quantification of the amplified product.

In a further aspect, the present invention provides a computer program for carrying the method for evaluating a property of a clinical treatment in a group of test subjects.

In a further aspect, the present invention provides a data carrier having a program saved thereon for carrying out the method for evaluating a property of a clinical treatment in a group of test subjects.

In a further aspect, the present invention provides a computer programmed to carry out the method for evaluating a property of a clinical treatment in a group of test subjects.

### Inhibitors

Compounds may be employed or screened for use in the present invention for treating a PPM1D-associated cancer. More particularly the compounds are inhibitors of PPM1D.

An example of a small molecule compound which is a PPM1D inhibitor and which may be used in accordance with the invention is SPI-001 (Yagi et al., Bioorg Med Chem Lett., January 1; 22(1), 729-32, 2012). A further example of a small-molecule PPM1D inhibitor is CCT007093 (Tan et al., Clin Cancer Res., April 15; 2269, 2009). Inhibitors of PPM1D may inhibit one or more activities of the polypeptide. For example, the inhibitors may inhibit phosphatase activity of the PPM1D polypeptide.

In addition the methods employed or screened for use disclosed herein may include the step of test candidate agents for binding to PPM1D using assays well known in the art.

Antibodies are an example of a class of inhibitor useful for treating a PPM1D-associated cancer, more particularly as inhibitors of PPM1D. Such antibodies may be useful in a therapeutic context (which may include prophylaxis).

Antibodies can be modified in a number of ways and the term “antibody molecule” should be construed as covering any specific binding member or substance having an antibody antigen-binding domain with the required specificity. Thus, this term covers antibody fragments (such as Fab, scFv, Fv, dAb, Fd; and diabodies) and derivatives, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP 0 120 694 A and EP 0 125 023 A.

Another class of inhibitors useful for treating a PPM1D-associated cancer includes peptide fragments that interfere with the activity of PPM1D. Peptide fragments may be generated wholly or partly by chemical synthesis, that block the catalytic sites of PPM1D. Peptide fragments can be readily prepared according to well-established, standard liquid and solid-phase peptide synthesis methods, general descriptions of which are broadly available (see, for example, M. Bodanzsky and A. Bodanzsky, The Practice of Peptide Synthesis, Springer Verlag, New York (1984); and Applied Biosystems 430A Users Manual, ABI Inc., Foster City, Calif.).

Other candidate compounds for inhibiting PPM1D may be based on modelling the 3-dimensional structure of these enzymes and using rational drug design to provide candidate compounds with particular molecular shape, size and charge characteristics. A candidate inhibitor, for example, may be a “functional analogue” of a peptide fragment or other compound which inhibits the component, with the same functional activity as the peptide or other compound in question.

Another class of inhibitors useful for treatment of a PPM1D-associated cancer includes nucleic acid inhibitors of PPM1D (NM_{—}003620), or the complements thereof, which inhibit activity or function by down-regulating production of active polypeptide. This can be monitored using conventional methods well known in the art, for example by screening using real time PCR.

Expression of PPM1D may be inhibited using anti-sense based technologies which engage RNA interference (RNAi). The use of these approaches to down-regulate gene expression is now well-established in the art. Construction of anti-sense sequences and their use is described for example in Peyman & Ulman, Chemical Reviews, 90:543-584, 1990 and Crooke, Ann. Rev. Pharmacol. Toxicol., 32:329-376, 1992. Methods relating to RNAi gene silencing are described for example in Fire, Trends Genet., 15: 358-363, 1999 and Elbashir et al, Nature, 411: 494-498, 2001.

Small RNA molecules may be employed to regulate PPM1D expression through RNAi. Methods that may be used to regulate PPM1D expression through RNAi include targeted degradation of mRNAs by small interfering RNAs (siRNAs) and short hairpin RNAs (shRNAs), post transcriptional gene silencing (PTGs), developmentally regulated sequence-specific translational repression of mRNA by micro-RNAs (miRNAs) and targeted transcriptional gene silencing. An example of a small RNA molecule inhibitor of PPM1D expression is described in Tan et al., Clin. Cancer Res., April 15; 2269, 2009.

Small RNA molecule PPM1D inhibitors may be produced within a cell, by in vitro transcription from a vector, or using standard solid or solution phase synthesis techniques which are known in the art. Linkages between nucleotides may be phosphodiester bonds or alternatives, e.g., linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through-O-or-S—.

Modified nucleotide bases can be used in addition to the naturally occurring bases, and may confer advantageous properties on siRNA molecules containing them (for example, increased stability). The term ‘modified nucleotide base’ encompasses nucleotides with a covalently modified base and/or sugar. Examples of modified nucleotide bases are known in the art.

In a further aspect, the present invention provides inhibitors for use in the method of treating a PPM1D-associated cancer.

### Pharmaceutical Compositions

The active agents for the treatment of PPM1D-associated cancer may be administered alone, but it is generally preferable to provide them in pharmaceutical compositions that additionally comprise with one or more pharmaceutically acceptable carriers, adjuvants, excipients, diluents, fillers, buffers, stabilisers, preservatives, lubricants, or other materials well known to those skilled in the art and optionally other therapeutic or prophylactic agents. Examples of components of pharmaceutical compositions are provided in Remington's Pharmaceutical Sciences, 20th Edition, 2000, pub. Lippincott, Williams & Wilkins.

These compounds or derivatives of them may be used in the present invention for the treatment of PPM1D-associated cancer. As used herein “derivatives” of the therapeutic agents includes salts, coordination complexes, esters such as in vivo hydrolysable esters, free acids or bases, hydrates, prodrugs or lipids, coupling partners.

The active agents disclosed herein for the treatment of PPM1D-associated cancer according to the present invention are preferably for administration to an individual in a “prophylactically effective amount” or a “therapeutically effective amount” (as the case may be, although prophylaxis may be considered therapy), this being sufficient to show benefit to the individual.

The agents for the treatment of PPM1D-associated cancer may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

Compositions comprising agents disclosed herein for the treatment of PPM1D-associated cancer may be used in the methods described herein in combination with standard chemotherapeutic regimes or in conjunction with radiotherapy. Examples of other chemotherapeutic agents include inhibitors of topoisomerase I and II activity, such as camptothecin, drugs such as irinotecan, topotecan and rubitecan, alkylating agents such as temozolomide and DTIC (dacarbazine), and platinum agents like cisplatin, cisplatin-doxorubicin-cyclophosphamide, carboplatin, and carboplatin-paclitaxel. Other suitable chemotherapeutic agents include doxorubicin-cyclophosphamide, capecitabine, cyclophosphamide-methotrexate-5-fluorouracil, docetaxel, 5-flouracil-epirubicin-cyclophosphamide, paclitaxel, vinorelbine, etoposide, pegylated liposomal doxorubicin and topotecan.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In a further aspect, the present invention provides pharmaceutical compositions for use in the method of treating a PPM1D-associated cancer.

### Materials and Methods

### Patients and Samples

### Cases

Lymphocyte DNA was used from 8,046 individuals affected with breast and/or ovarian cancer that were recruited via two studies. 7,724 cases were recruited through 24 genetics centres in the UK via the Breast and Ovarian Cancer Study (BOCS), which recruits women ≧18 years who have had breast cancer and/or ovarian cancer and have a family history of breast cancer and/or ovarian cancer. Each proband was screened for BRCA1 and BRCA2 mutations (by Sanger sequencing and/or heteroduplex analysis) and large rearrangements (by MLPA). The remaining 322 cases are an unselected hospital-based series of women with ovarian cancer who were recruited during treatment for ovarian cancer at the Royal Marsden Hospital. The DNA was extracted from peripheral blood samples except in 11 cases, for whom DNA was extracted from a lymphoblastoid cell line (NB all the PPM1D mutations were identified in peripheral blood-derived DNA). At least 97% of families were of European ancestry, i.e. comparable to the controls. Informed consent was obtained from all participants. The research was approved by the London Multicentre Research Ethics Committee (MREC/01/2/18).

For the Phase 1 pooled DNA repair panel experiment lymphocyte DNA was used from 1,150 women with breast cancer, 69 also had ovarian cancer. 78 of these individuals had one mutation, and one individual had two mutations, in known cancer predisposition genes. These were included as ‘positive controls’ to evaluate variant calling (see below). For the PPM1D case-control sequencing experiment 7,781 individuals with breast and/or ovarian cancer were used. The case data from the pooled DNA repair panel experiment was not used in the case-control analysis, firstly because the mutation status of individuals cannot be definitively obtained from the pooled experiment as one cannot be certain that every sample is equally represented in a pool, and secondly because the mutation detection method was different to that utilised in the case-control experiment. Standard case and control sample trays were used for the case-control PPM1D sequencing experiment and the sample selection was blind to the pooled DNA repair panel experiment. 885 individuals were part of both experiments.

Samples and Pathology Information from Mutation-Positive Families

For families in which a PPM1D mutation was detected, DNA samples were obtained from relatives. Tumour material, pathology information, and receptor status in probands was requested from the hospitals where the individuals had been treated. Representative tumour blocks were retrieved where possible and examined by two histopathologists (DNR & JSR-F) and classified and graded according to the World Health Organisation 2003 classification. Tumours were microdissected under a stereomicroscope and genomic DNA was extracted from tumour and, where possible, stroma using the DNeasy kit (Qiagen).

### Controls

Lymphocyte DNA was used from 5,861 population-based controls obtained from the 1958 Birth Cohort Collection, an on-going follow-up of persons born in Great Britain in one week in 1958. Biomedical assessment was undertaken during 2002-2004 at which blood samples and informed consent were obtained for creation of a genetic resource but phenotype data for these individuals is not available. At least 97% of the controls were of European ancestry. (http://www/cls.ioe.ac.uk/studies.asp?section=000100020003).

### Sequencing

### DNA Repair Panel Sequencing

Genes for inclusion on the DNA repair panel were identified from http://www.geneontology.org/ using the search term “DNA repair” (GO:0006281) and from http://string-db.org/ by identifying all genes interacting with ATM, BRCA1, BRCA2, BRIP1, CHEK2 and PALB2 with highest confidence (≧0.9). This dataset was manually curated to remove duplicate genes and pseudogenes. CCDS transcripts for the remaining genes were retrieved from UCSC Genome Browser (http://genome.ucsc.edu/ from November 2010). Genomic coordinates for all coding exons were identified and targeted in a custom pulldown designed using the Agilent SureSelect Target Enrichment system (Agilent)¹. 48 pools of DNA were created, that each included 4 μl of 50 ng/μl=200 ng of DNA from 24 individuals. 80 μl of the pooled DNA was sheared using Covaris technology. Libraries were prepared without gel size selection or PCR enrichment using the Illumina Genomic PE Sample Prep Kit (Illumina) and target enrichment was performed according to the Agilent SureSelect protocol. Sequencing was performed by the WTCHG High-throughput DNA sequencing and MRC hub in Oxford on an Illumina HiSeq2000 (v2 flow cell, one lane of sequencing per pool) generating 2×100 bp reads. Sequence reads for each pool were mapped to the human reference genome (hg19) using BWA (version 0.5.6)². Mapped reads were filtered to remove ambiguous alignments with a quality score of 0 and bases with a call quality below 22 were masked. Of the remaining reads for each pool 50-60% fell within the target regions, except for Pool 21 where the on target percentage was significantly lower. Median coverage for each pool achieved for target regions after filtering was between 2849× and 5545×. This corresponded to an average coverage of 119×-231× per sample. All pools had 90% of the target covered at a minimum of 480×. Target regions within the MHC achieved substantially lower coverage and were excluded from further analysis.

The DNA repair panel was also sequenced in six PPM1D PTV positive individuals using Illumina TruSeq kits for library preparation to enable sample indexing. Genomic DNA (1.5 μg) was fragmented and the libraries prepared using the Illumina TruSeq Sample Preparation Kit (index set A). One pool of six libraries (500 ng each) was enriched as before but with the addition of extra blocking primers targeted against the TruSeq index adapter sequences. Sequencing was performed at ICR with an Illumina HiSeq2000 (v3 flowcell, one lane) generating 2×100 bp reads. Mapped reads were filtered to remove ambiguous alignments with a quality score of 0 and bases with a call quality below 22 were masked. Of the remaining reads, 41-43% fell within the target region for each individual. Median coverage of the target for each individual after filtering was between 602× and 690×. All individuals had 90% of the target covered at a minimum of 50×.

### PPM1D Sanger Sequencing

Primers were designed to PCR amplify and Sanger sequence PPM1D using Exon-Primer from UCSC Genome Browser (http://genome.ucsc.edu/ from November 2010). Primers and conditions are available on request. PCR reactions were performed using the QIAGEN Multiplex PCR Kit (Qiagen). Amplicons were unidirectionally sequenced using the BigDye Terminator Cycle sequencing kit and an ABI3730 automated sequencer (ABI PerkinElmer). The full coding sequence was analysed in 2,456 cases and 1,347 controls. As all the mutations identified in these samples were restricted to exon 6, the mutation cluster region (c.1261-20-c.1695) was sequenced, but not the rest of the gene, in the remaining 5,325 cases and 4,514 controls. The mutation cluster region was also sequenced in all available samples from relatives of PPM1D PTV positive probands. All sequencing traces were independently analysed by two individuals who were blind to the others analysis. Each individual analysed the sequencing with both automated software (Mutation Surveyor, SoftGenetics) and manual visual inspection. All putative mutations were confirmed by bidirectional sequencing from a fresh aliquot of the stock DNA. Sanger sequencing of the PPM1D cluster region was also performed, in triplicate, in DNA from eight tumour samples and four ovarian stromal samples.

For the cDNA sequencing, lymphoblastoid cell lines were established from three individuals with PPM1D PTVs (cases 20, 23 and 24). RNA was extracted using RNeasy Minikit (Qiagen) and cDNA synthesised using the ThermoScript RT-PCR system (Invitrogen), employing standard protocols. The mutation cluster region was amplified using a cDNA-specific primer, [Forward_ACCACCAGTCAAGTCACTGG; Reverse_TCTTTCGCTGTGAGGTTGTG] which was sequenced as described above.

### Deep PCR Amplicon Sequencing

The PPM1D mutation cluster region, full coding sequence and intron-exon boundaries of BRCA1 and BRCA2 was amplified from lymphocyte DNA using the Multiplex PCR Kit (Qiagen). Indexed libraries of the PCR products were prepared using Nextera technology (Illumina)³. Two pools of 24 indexed libraries were created which were subsequently sequenced using an Illumina MiSeq, generating 2×150 bp reads. Data from 20 individuals passed quality control coverage metrics, generating median coverage greater than 500× across the PPM1D cluster region (average median coverage 3384×).

For the tumour analyses, the mutation cluster region was amplified in tumour, stroma and blood DNA using an Illumina Nextera XT library preparation kit and supplied protocol (Illumina). To attain the required 1 ng input for tagmentation BRCA1 was also amplified in 24 samples as described above and then one pool of 24 indexed libraries was created, which was then sequenced using an Illumina MiSeq, generating 2×150 bp reads. Sequencing reads present at the mutation site were visually inspected after alignment with Stampy to determine if the PPM1D mutation was present.

### NGS Data Analysis

### DNA Repair Panel Data

For the pooled DNA repair panel analysis, variant calling was undertaken with Syzygy (version 1.2.4)⁴. 402/439 previously validated SNPs with a MAF>5% genotyped through a breast cancer GWAS were successfully identified with high confidence and the remaining 37 SNPs were detected at lower confidence. Syzygy also detected 75/80 rare variants (MAF<1%) included in the study as positive controls (24/26 base substitutions, 14/14 insertions, 30/32 deletions and 7/8 complex indels). Thus sensitivity was 99.6% for base substitutions and 94.4% for rare indels. Frequency estimation for rare variants was assessed by evaluation of 39 BRCA1 and BRCA2 variants at a frequency of one per pool. Syzygy correctly estimated the frequency in 33 of the 35 variants it detected, incorrectly estimating the frequency at two per pool for the remaining two variants.

### Deep PCR Amplicon Sequencing Data

For the deep PCR amplicon sequencing and the indexed DNA repair panel sequencing in six individuals, sequence reads were mapped to the human reference genome (hg19) using Stampy version 1.0.14⁵. Duplicate reads were flagged using Picard version 1.60 (http://picard.sourceforge.net). Variant calling was performed with Platypus version 0.1.9 (http://www.well.ox.ac.uk/platypus). The mutant read percentage was calculated as the proportion of total reads at the variant location that contained the variant, with a minimum mutant read percentage threshold of 5%.

### Variant Annotation

Annotation for all experiments was undertaken with reference to CCDS transcripts from EnsEMBL version 65 identified using a custom Perl script. Variant calls were annotated for changes with respect to the chosen transcript and assigned a consequence type from the list used by EnsEMBL.

### PTV Prioritisation Method

This is a gene-based (rather than the more typical variant-based) strategy that aims to prioritise potential disease-associated genes for follow-up by leveraging two properties of protein truncating variants: (1) the strong association of rare truncating variants with disease, and (2) collapsibility; different PTVs within a gene typically result in the same functional effect and can be combined equally. The method was implemented in the statistical software package R. All the predicted protein truncating variants were first outputted: stop gains, coding frameshifts and essential splice site variants (−2, −1, +1, +2, +5). For this experiment ‘rare’ variants were defined as PTVs that were seen only once in the DNA repair panel data. The genes were then stratified according to the number of different, rare singleton PTVs called. Genes for which samples had been included as positive controls were excluded. PPM1D was the top gene in this analysis.

### MLPA

22 probe pairs were designed, targeting PPM1D PTVs (n=18), wildtype PPM1D (n=2), wildtype BRCA1 (n=1) and wildtype CEP112 (n=1). The synthetic probes were added to the SALSA MLPA probe mix P200 (MRC Holland). MLPA reactions were performed in triplicate according to the manufacturer's instructions. MLPA was undertaken in lymphocyte DNA from 17 probands and in eight tumour DNA samples (from five individuals). In brief, probes were hybridised to 150 ng of denatured DNA, amplified by PCR, and separated on an ABI 3130 Genetic Analyzer (Applied Biosystems). Data were analysed using GeneMarker v1.51 software (SoftGenetics).

### Microsatellite Analysis

5′6-FAM tagged primer pairs and PCR conditions were used for 17q microsatellite analysis. 10 μl of a mastermix of 30 μl ROX size standard and 1 ml HiDi formamide were added to each reaction post PCR, denatured at 95° C. for 5 minutes, and cooled at −20° C. for 5 minutes. Reactions were run on a 3730×L genetic analyser (Applied Biosystems) under the fragment analysis protocol. Data were analysed using GeneMarker v1.51 software (SoftGenetics). Microsatellite analysis was undertaken in lymphocyte DNA from 13 individuals from eight families, and in eight tumour DNA samples and four stroma DNA samples from five individuals. Of note, one of these cases (17) harbours both BRCA1 and PPM1D mutations. Both genes are located at chromosome 17q and it is the wild-type BRCA1 allele that is reduced in the tumours and therefore the relevance of the loss of heterozygosity with respect to PPM1D is difficult to deduce.

### Cell Line and Plasmid Constructs

The U2OS (p53 wildtype) cell line was obtained from the American Type Culture Collection (ATCC). Cells were cultured and maintained according to the supplier's instructions. Cells were transfected with plasmid DNA using Lipofectamine 2000 (Invitrogen). A plasmid containing full-length wildtype PPM1D cDNA (pCMV6 entry-PPM1D) was obtained from Origene, and the PPM1D open reading frame (ORF) subcloned into pCMV6-AN-HA (Origene), generating a construct that could express a PPM1D-N-terminal HA epitope fusion protein. Truncating mutations were introduced into the PPM1D ORF of this construct using the QuickChange II XL Site-Directed Mutagenesis Kit (Stratagene). To generate the following mutants, the following DNA amplification primers were used:

| PPM1D mutant 1 (c.1384C > T), | |
|---|---|
| forward primer | GAGAGAATGTCTAAGGTGTAGTC, |
| reverse primer | GACTACACCTTAGACATTCTCTC, |
| PPM1D mutant 2 (c.1420delC), | |
| forward primer | GATCCAGAACCATTGAAG, |
| reverse primer | CTTCAATGGTTCTGGATC. |

### Western Blot Analysis of P53 Levels

U2OS cells were transfected with PPM1D expression constructs and 24 hours after transfection, cells were exposed to gamma irradiation (5 Gy) from an X ray source. Whole cell lysates were generated from transfected cells after irradiation (at 30 minute and four hour time points) and subjected to protein electrophoresis. Immunoblotting of electrophoresed lysates was performed using antibodies specific for p53 (9282S—Cell Signaling Technology) and actin (sc-1616, Santa Cruz Biotech).

### Frequency and Risk Estimation

Statistical analyses were performed using the statistical package R. The significance of mutation clustering was modelled under a binomial distribution where the probability of observing a mutation in the last exon, which comprises 31% of the coding sequence, was 0.31. The frequency in BRCA1/BRCA2 carriers and non-carriers was compared using a two-sided test of proportions. Risk estimation was implemented using a competing risks retrospective likelihood model incorporating age at onset according to a proportional hazards model. Since individuals screened for PPM1D mutations were selected on the basis of both personal and family history of breast or ovarian cancer, standard methods of analysis that ignore the sampling frame would yield biased estimates of the risk ratios. To address this, the data were analysed within a retrospective cohort approach by modelling the conditional likelihood of the observed genotypes given the disease phenotypes, using information on breast and ovarian cancer occurrence in the set of 6,577 unrelated individuals negative for BRCA1/2 mutations (BRCA1/2 mutation-positive individuals from the FBCS series and all the unselected ovarian case series were excluded). A competing risks model was assumed, under which, each individual was at risk of developing breast or ovarian cancer. This provides unbiased estimates of the risk ratios for breast and ovarian cancer where a genetic variant may be associated with one or both of the diseases. The PPM1D mutation carrier frequency in the population and breast and ovarian cancer risk ratios were estimated simultaneously. Since mutation screened probands may have been selected on the basis of bilateral breast cancer diagnosis or on the basis of both breast and ovarian cancer diagnosis the risks of breast or ovarian cancer diagnosis after the first cancer diagnosis were allowed for, including the risk of contralateral breast cancer. This model assumes that the increased breast cancer (including contralateral) or ovarian cancer risk after the first cancer diagnosis is entirely due to the susceptibility as defined by the model, with no additional variation in risk. Site-specific cancer risks were assumed to be independent conditional on genotype. Therefore the incidence of cancer at the second site was assumed to be the same as if the preceding cancer had not occurred, with the exception of contralateral breast cancer incidence after the first breast cancer, which was assumed to be half the overall breast cancer incidence, since only one breast was at risk. In all models females were censored at age 80 years. Breast and ovarian cancer incidences were assumed to be dependent on the underlying PPM1D genotype through models of the form: λ(t)=λ₀(t)exp(β_{χ}) where λ₀(t) is the baseline incidence at age t in non-mutation carriers, β is the log risk ratio associated with the mutation and χ takes value 0 for non-mutation carriers and 1 for mutation carriers. The overall breast and ovarian cancer incidences, over all genotypes, were constrained to agree with the population incidences for England and Wales in the period of 1993-1997. The models were parameterised in terms of the mutation frequencies and log-risk ratios for breast and ovarian cancer. Parameters were estimated using maximum likelihood estimation and were implemented in the pedigree analysis software MENDEL⁶. The variances of the parameters were obtained by inverting the observed information matrix. To obtain confidence intervals for the risk ratios and perform hypothesis testing, log risk ratios were assumed to be normally distributed. A Wald test-statistic was used to test the null hypothesis that β=0 for both breast and ovarian cancer. Since PPM1D mutations were not found to segregate within families, precise family histories or pedigree information was not taken into account and therefore did not incorporate the effects of other susceptibility genes.

### Results/Discussion

To investigate the role of DNA repair genes in cancer susceptibility, 507 genes (the ‘DNA repair panel’) were sequenced in 1,150 individuals with breast cancer from the UK, 69 of whom also had ovarian cancer (Table 2). To maximise time, sample and cost efficiency a pooled approach was used, combining 200 ng of DNA from each of 24 individuals into a single pool which were hybridised to a custom pulldown containing the DNA repair panel. Sequencing was performed using an Illumina HiSeq2000 which generated a minimum coverage per pool of 480× for 90% of the target region. Sequence variants were called using Syzygy⁴, the performance of which was evaluated using previously generated data in a subset of the samples. The sensitivity of base substitution calling was 99.6% (439/439 common variants and 24/26 rare variants that were present in 1/24 individuals in a pool). The sensitivity of insertion/deletion calling was 94.4% (51/54 rare insertion/deletions present in 1/24 individuals in a pool).

The 34,564 sequence variants called by Syzygy were next considered. PTVs were focussed on first because of the strong association of this class of mutation with disease. In total, 1,044 PTVs were called by Syzygy and a ‘PTV prioritisation method’ was used to stratify the genes according to the number of different, rare truncating mutations present within the samples⁷. PPM1D showed the strongest signal in this analysis, and Sanger sequencing was used to confirm that five individuals carried different PPM1D PTVs. Two of these individuals had ovarian cancer in addition to breast cancer.

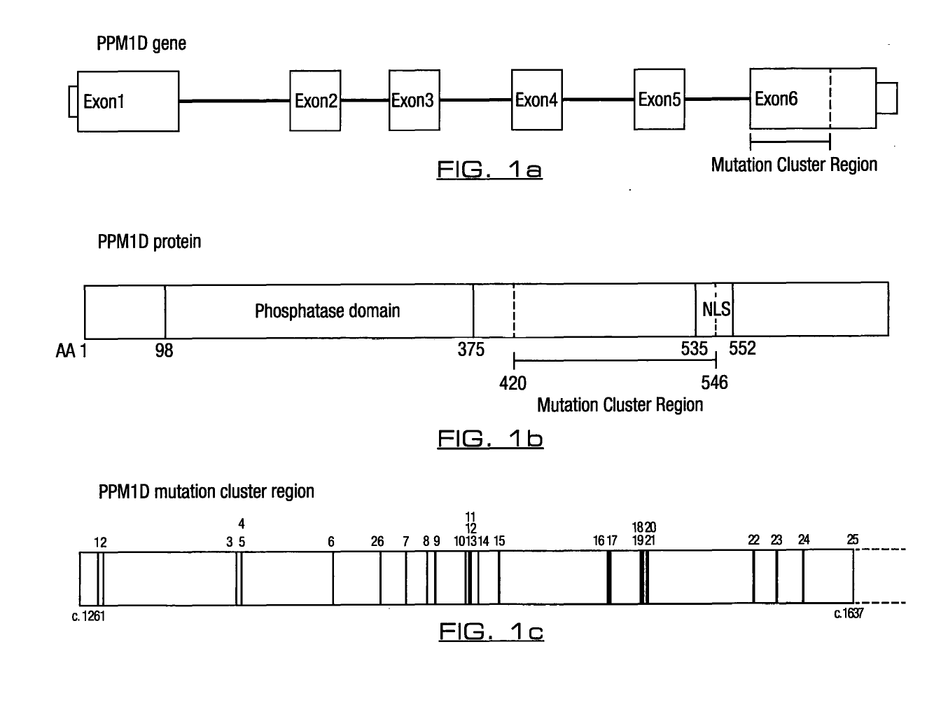

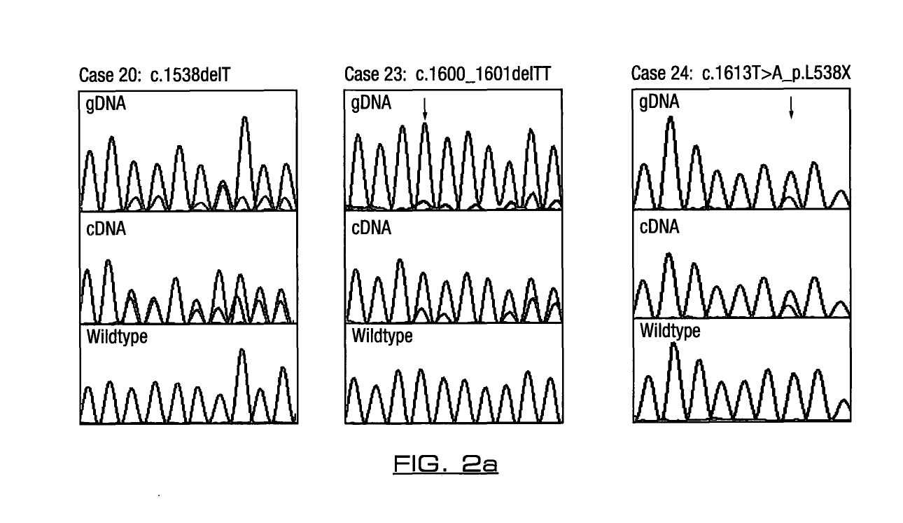

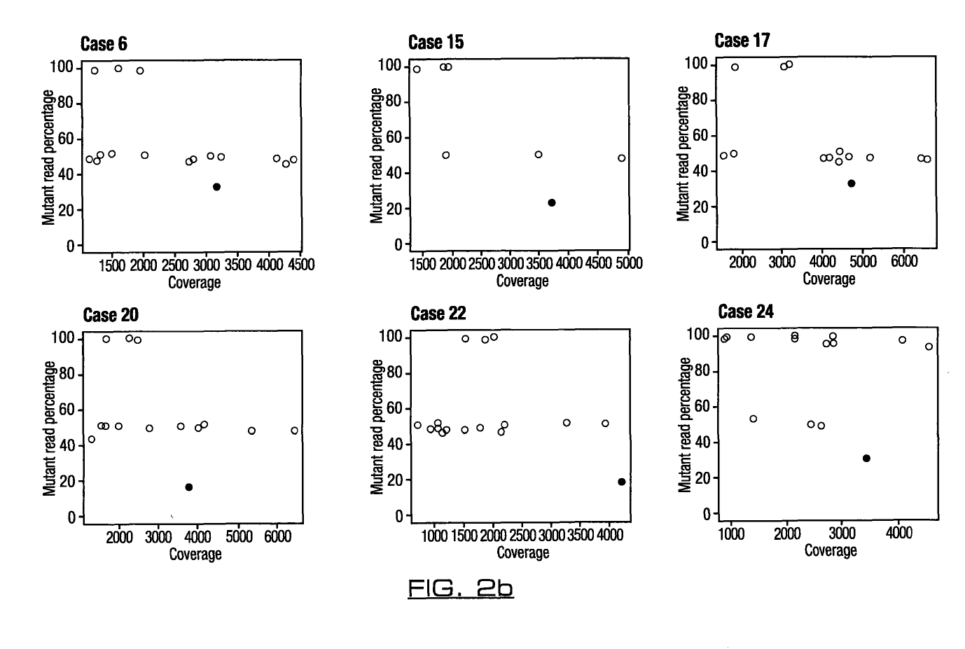

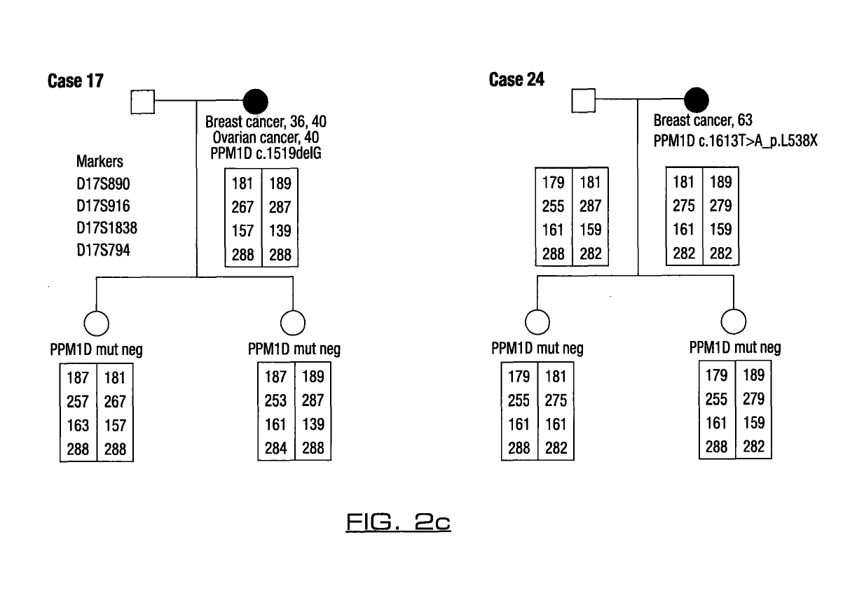

To further explore the role of PPM1D in breast and ovarian cancer susceptibility, a case-control Sanger sequencing analysis of PPM1D was performed in a total of 13,642 individuals; 7,781 unrelated individuals with breast and/or ovarian cancer and 5,861 population controls (Table 2). Initially, all PPM1D exons and intron-exon boundaries were sequenced but after completing this analysis in 3,803 samples it was noted that all 10 PTV mutations identified occurred within the last exon of PPM1D, and that this clustering was highly significant (P=8.2×10⁻⁶). The remaining 9,839 samples were thus analysed for this mutation cluster region (MCR), identifying a further 16 PTVs (Table. 1, FIG. 1). It total 25 PPM1D PTVs were identified in individuals with breast and/or ovarian cancer, and 1 was found in controls (P=1.12×10⁻⁵, FIG. 1, FIG. 2a and Table 3). This included 18 mutations in 6,912 individuals with breast cancer (P=2.42×10⁻⁴) and 12 mutations in 1,121 individuals with ovarian cancer (P=3.10×10⁻⁹). The histological features of the cancers in PPM1D mutation carriers were diverse, and five individuals had both breast and ovarian cancer. The case series included 773 individuals with mutations in BRCA1 or BRCA2 (termed ‘BRCA1/2 mutation carriers’), four of whom also carried PTVs in PPM1D (4/773 vs. 1/5861 controls, P=8.30×10⁻⁴). A total of 16 non-synonymous, 14 synonymous and one intronic variant were also found across the cases and controls; there was no evidence for an association with cancer for these variant classes.

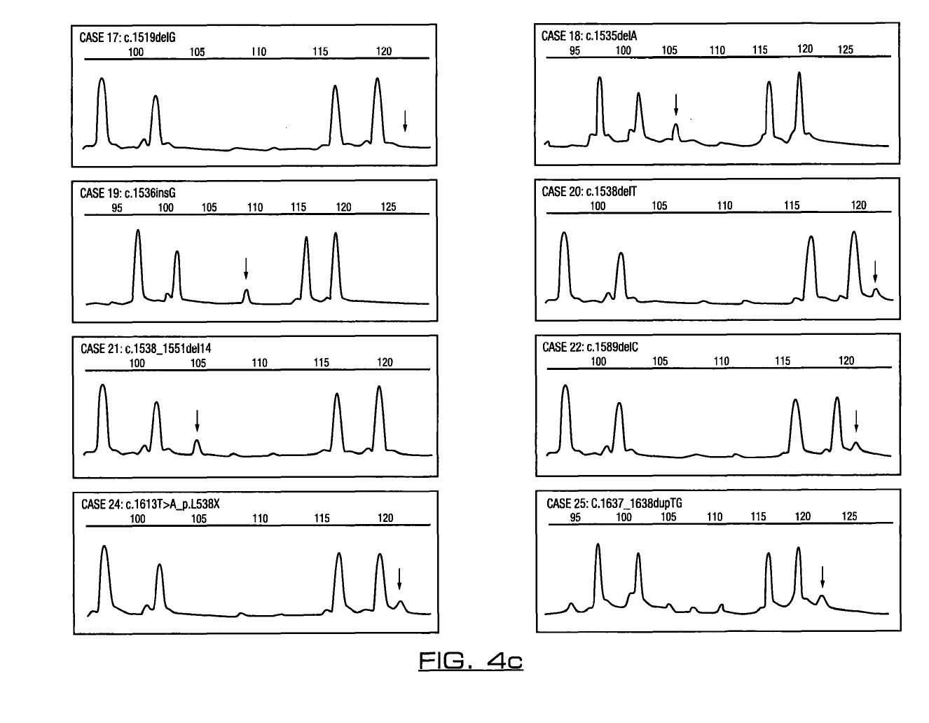

Sanger sequencing chromatograms for the PPM1D PTVs were unusual for heterozygous mutations as the mutant allele was considerably and consistently lower than the wildtype allele, suggesting the mutations were mosaic in lymphocyte DNA (FIG. 2a). DNA from saliva was available for two individuals and the PTVs were present at similar amplitude to that identified in the corresponding blood derived DNA. To further confirm the PTV mutations were bona fide two additional mutation detection methods were used; deep PCR amplicon sequencing⁸ (FIG. 2b, and Table 3) and multiplex ligation-dependent probe amplification (MLPA)⁹ (FIG. 4). For the deep PCR amplicon sequencing Nextera libraries of pooled PCR products covering BRCA1, BRCA2 and the PPM1D mutation were generated and sequenced using an Illumina MiSeq, generating a median coverage of 3387× across the PPM1D mutation (Table 3). This confirmed the PPM1D PTVs were present at a lower proportion than heterozygous polymorphisms in BRCA1 and BRCA2, with a median mutant read percentage of 16% (range 5-34%; FIG. 2b). Additionally, the original DNA repair panel was sequenced in six cases individually (i.e. unpooled), which confirmed the mutations were present, but mosaic (Table 3). For three samples data from both the deep PCR amplicon sequencing and the DNA repair panel were available, and gave identical mutation percentage results (Table 3). Finally, family studies were also consistent with mosaicism; none of 14 relatives carried the PPM1D mutation identified in the proband. For each of probands 17 and 24, two offspring were identified that had inherited different maternal haplotypes at the PPM1D locus, but neither offspring carried the relevant maternal PPM1D mutation, demonstrating that the mutations were either not present, or mosaic in the germline of the probands (FIG. 2c).

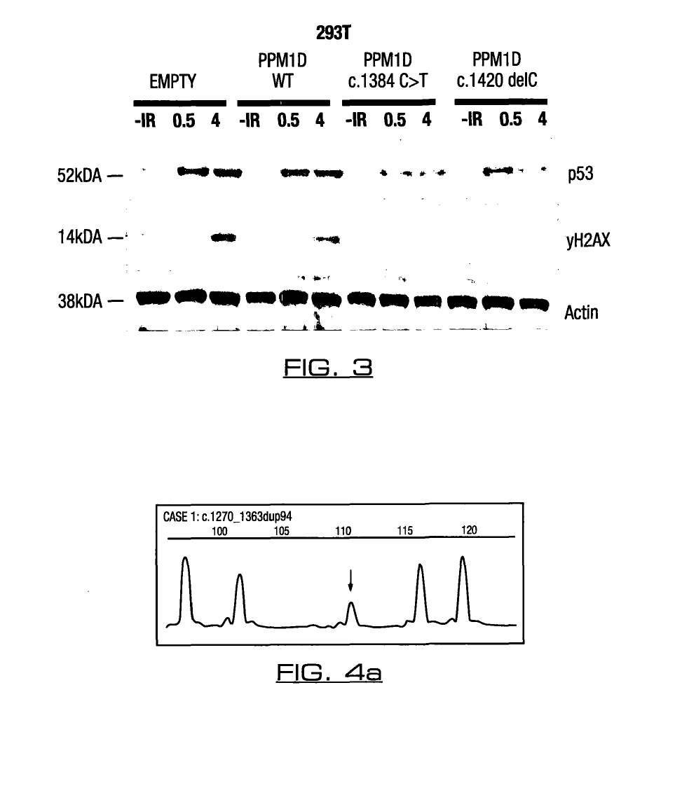

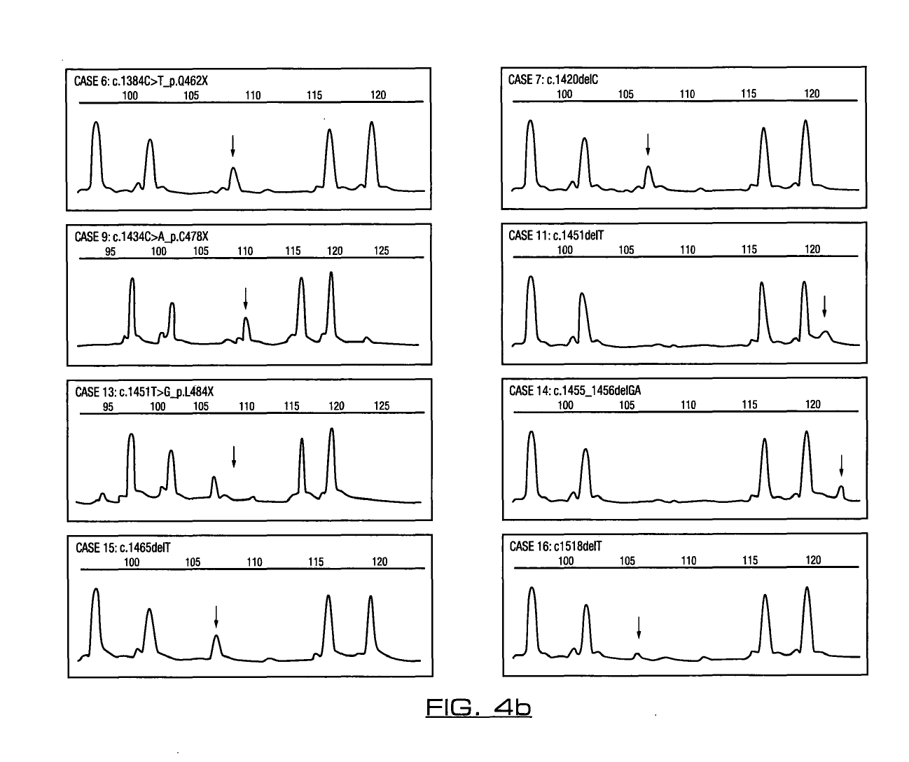

The clustering of PTVs within the 370 bp region corresponding to amino acids 420-546, which is downstream of the phosphatase catalytic domain but precedes or disrupts the nuclear localisation signal, suggested the PTVs were not acting as simple loss-of-function mutations (FIG. 1). Moreover, all the PTVs were in the last exon and thus predicted to evade nonsense-mediated RNA decay and to result in a truncated protein that retains the phosphatase catalytic domain, rather than in haploinsufficiency. This was confirmed this experimentally for three mutations (FIG. 2a). To investigate the effect of PPM1D PTVs cDNA expression constructs representing two mutant alleles (PPM1D c.1384C>T; case 6 and PPM1D c.1420delC; case 7) were generated and tested for their ability to suppress p53 activation in response to ionising radiation (IR) exposure. Normal elevation of p53 levels after IR exposure was moderately suppressed in human U2OS tumour cells transfected with a wildtype PPM1D expression construct, matching previous observations (FIG. 3). The suppression of p53 was enhanced in cells transfected with the mutant PPM1D expression constructs suggesting that each of these alleles encodes a hyperactive PPM1D isoform, i.e. consistent with a gain-of-function rather than a loss-of-function effect (FIG. 3).

To investigate the mechanism of oncogenesis in PPM1D PTV mutation carriers, eight tumours were analysed from five individuals. The PPM1D mutations were not detectable in any of the tumours by Sanger sequencing or MLPA. Through microsatellite analysis the tumours were confirmed to be from the correct individuals and loss of heterozygosity was demonstrated at the PPM1D locus in seven of eight tumours, though there was no evidence of PPM1D copy number alteration. Stromal tissue was microdissected from the ovarian tumour in four cases and the PPM1D PTV was deep sequenced in blood, tumour and stromal DNA. Each mutation was present in the blood, at similar level to that detected previously, absent from the tumour and either absent (two cases) or present at very low level (5/915 reads and 4/5793 reads) in the stroma, consistent with lymphocyte contamination.

These data strongly suggest the mechanism of cancer association in PPM1D mutation carriers differs from that in carriers of mutations in other DNA repair genes associated with predisposition to these cancers. Without wishing to be bound by any particular theory, there are several potential explanations. For example, it is possible the mutation was present in the cell of cancer origin but was subsequently lost, perhaps because a PPM1D mutation acts only as a driver to initiate oncogenesis. Alternatively, the absence of the PPM1D mutation in the tumour could be because oncogenesis is being driven by the mutation in circulating blood cells.

Irrespective of the mechanism of the association, the present invention demonstrates that individuals with PPM1D PTVs in the mutation cluster region are at increased risk of cancer. To estimate the cancer risks a retrospective cohort analysis was undertaken, modelling the retrospective likelihood of the observed mutation status conditional on the disease phenotype, as previously described¹⁰. This approach adjusts for the ascertainment of cases with more extreme phenotypes such as young age of onset, bilateral breast cancer and/or family history of cancer, which are used to empower gene discovery^{10,11}. The relative risk of breast cancer for PPM1D PTV carriers was estimated to be 2.7 (95% CI: 1.3-5.3; P=5.38×10⁻³), which translates to approximately 23% cumulative risk by age 80. The relative risk of ovarian cancer was estimated to be 11.5 (95% CI: 4.3-30.4; P=9.95×10⁻⁷), which translates to approximately 18% cumulative risk by age 80. It is noteworthy that an unselected hospital-based series of 322 ovarian cancer patients was included in whom five PPM1D PTVs were identified, suggesting that 1-2% of ovarian cancer patients may harbour mosaic PPM1D mutations.

The frequency of PPM1D PTVs in BRCA1/2 mutations carriers with breast and/or ovarian cancer was also significantly different from population controls (4/773 vs. 1/5861; P=8.30×10⁻⁴) and similar to that in cases of breast and/or ovarian cancer without BRCA1/2 mutations (4/773 vs. 21/6634; P=0.56), suggesting that PPM1D PTVs are also associated with increased risks of cancer in BRCA1/2 mutation carriers. Studies of unselected, population-based cancer patients and of larger series of BRCA1/2 mutation carriers would be of value to extend the observations of the present study, and to further explore the prevalence and cancer risks associated with PPM1D mutations.

The present invention provides new insights into ovarian and breast cancer, identifying a novel class of genetic defect that lies somewhere between classic germline genetic predisposition mutations and tumour-specific somatic events. It is also likely that PPM1D mutations are associated with other cancers, and broad evaluation of individuals with other tumour types would be of interest. The clinical implications of a mosaic cancer predisposition marker that is genetic, but not hereditary, and that is detectable in the blood but not the tumour(s) it is associated with are rather profound.

Moreover, the present invention provides insights into genetic variation, particularly in that rare and mosaic gene mutations can have relevance to common disease. Such variants are challenging to detect by Sanger sequencing, but are detectable by next-generation sequencing approaches. Although newer sequencing technologies are making large-scale whole-genome sequencing experiments ever more feasible, focussed sequencing experiments with tailored design and analytical prioritisation strategies, such as those employed herein, are required to ensure the implications of such variants in case series are correctly interpreted.

TABLE 1
PPM1D mutations and cancer phenotype

| ID | PPM1D mutations | Cancer (age in yrs) |
|---|---|---|
| 1^{a} | c.1270_1363dup94 | Ov ca (64), Bil br ca (43, 56) |
| 2 | c.1272delGGinsC | Br ca (34) |
| 3^{a} | c.1337C>G_p.S446X | Ov ca (43), Bladder ca (55) |
| 4^{a} | c.1340delA | Br ca (46) |
| 5 | c.1340delA | Br ca (65) |
| 6 | c.1384C>T_p.Q462X | Br ca (59) |
| 7 | c.1420delC | Ov ca (68), Br ca (71) |
| 8 | c.1430delA | Br ca (44) |
| 9 | c.1434C>A_p.C478X | Br ca (40) |
| 10 | c.1448delC | Br ca (41) |
| 11 | c.1451delT | Ov ca (67) |
| 12 | c.1451delT | Bil br ca (61, 76) |
| 13 | c.1451T>G_p.L484X | Br ca (65) |
| 14 | c.1455_1456delGA | Br ca (70) |
| 15 | c.1465delT | Ov ca (60), Bil br ca (50, 55) |
| 16 | c.1518delT | Ov ca (67) |
| 17^{b} | c.1519delG | Ov ca (40), Bil br ca (36, 40) |
| 18 | c.1535delA | Br ca (46) |
| 19 | c.1536insG | Ov ca (47) |
| 20 | c.1538delT | Ov ca (60), Br ca (55) |
| 21 | c.1538_1551del14 | Ov ca (41) |
| 22 | c.1589delC | Ov ca (69), Colorectal (69) |
| 23 | c.1600_1601delTT | Br ca (62) |
| 24 | c.1613T>A_p.L538X | Br ca (63) |
| 25 | c.1637_1638dupTG | Ov ca (76) |
| 26 | c.1412delC | control |

Ov ca, ovarian cancer;
br ca, breast cancer;
bil br ca, bilateral breast cancer.

TABLE 2
Summary of samples and PPM1D mutation status

| | | |
|---|---|---|
| Total number | Individuals | |
| of | with PPM1D | |
| individuals | mutation | P value^{a} |

Phase 1 - DNA repair panel sequencing

| | | |
|---|---|---|
| all cases | 1150^{b} | 5 |
| breast and ovarian cancer | 69 | 2 |

Phase 2 - case-control PPM1D sequencing

| | | | |
|---|---|---|---|
| all controls | 5861 | 1 | |
| full gene sequencing | 1347 | 0 | |
| MCR sequencing | 4514 | 1 | |
| all cases | 7781 | 25 | 1.12 × 10⁻⁵ |
| full gene sequencing | 2456 | 10 | |
| MCR sequencing | 5325 | 15 | |
| breast cancer cases | 6912 | 18 | 2.42 × 10⁻⁴ |
| ovarian cancer cases | 1121 | 12 | 3.10 × 10⁻⁹ |
| breast and ovarian | 252 | 5 | |
| cancer^{c} | | | |
| bilateral breast cancer^{d} | 886 | 4 | |
| unselected ovarian | 322 | 5 | |
| cancer case series^{e} | | | |
| BRCA1/2 mutation | 773 | 4 | 8.30 × 10⁻⁴ |
| carrier | | | |
| BRCA1 mutation^{f} | 364 | 1 | |
| BRCA2 mutation^{f} | 409 | 3 | |
| BRCA1/2 neg^{g} | 6634 | 21 | 3.66 × 10⁻⁵ |

^{a}P value is calculated from Fisher's exact test compared with controls
^{b}The samples were pooled and thus the exact number of individuals successful analysed is not known
^{c}These are also included in breast cancer cases and in ovarian cancer cases
^{d}These are also included in breast cancer cases
^{e}These are also included in the ovarian cancer cases
^{f}These are also included in the BRCA1/2 mutation carriers
^{g}This does not include 374 individuals for whom BRCA1/2 status is unknown (none carried a PPM1D mutation)

TABLE 3
Mutation analysis in 25 PPM1D carriers

| | PPM1D mutation | | BRCA mutation | | |
|---|---|---|---|---|---|
| ID | Nucleotide | Protein | Nucleotide | Protein | PPM1D MLPA |
| 1 | c.1270_1363dup94 | | BRCA2 c.7063G > T | p.E2355X | mut present |
| 2 | c.1272delGGinsC | | no mutation | | |
| | | | BRCA2 | | |
| 3 | c.1337C > G | p.S446X | c.5350_5351delAA | | |
| 4 | c.1340delA | | BRCA2 c.7558C > T | p.R2520X | |
| 5 | c.1340delA | | no mutation | | |
| 6 | c.1384C > T | p.Q462X | no mutation | | mut present |
| 7 | c.1420delC | | no mutation | | mut present |
| 8 | c.1430delA | | no mutation | | |
| 9 | c.1434C > A | p.C478X | no mutation | | mut present |
| 10 | c.1448delC | | no mutation | | |
| 11 | c.1451delT | | no mutation | | mut present |
| 12 | c.1451delT | | no mutation | | |
| 13 | c.1451T > G | p.L484X | no mutation | | mut present |
| 14 | c.1455_1456delGA | | no mutation | | mut present |
| 15 | c.1465delT | | no mutation | | mut present |
| 16 | c.1518delT | | no mutation | | mut present |
| 17 | c.1519delG | | BRCA1 c.2475delC | | mut present |
| 18 | c.1535delA | | no mutation | | mut present |
| 19 | c.1536insG | | no mutation | | mut present |
| 20 | c.1538delT | | no mutation | | mut present |
| 21 | c.1538_1551del14 | | no mutation | | mut present |
| 22 | c.1589delC | | no mutation | | mut present |
| 23 | c.1600_1601delTT | | no mutation | | |
| 24 | c.1613T > A | p.L538X | no mutation | | mut present |
| 25 | c.1637_1638dupTG | | no mutation | | mut present |

| | Deep PCR Amplicon Sequencing | | | | Individual DNA Repair Panel | | | |
|---|---|---|---|---|---|---|---|---|
| | | WT | Mutant | Mutant | | WT | Mutant | Mutant |
| ID | Coverage | Reads | Reads | Read % | Coverage | Reads | Reads | Read % |
| 1 | 2924 | 2786 | 138 | 5 | | | | |
| 2 | 2071^{a} | 1594^{a} | 477^{a} | 23^{a} | | | | |
| 3 | 3694 | 2693 | 1001 | 27 | | | | |
| 4 | 1715 | 1314 | 401 | 23 | | | | |
| 5 | 535 | 499 | 36 | 7 | | | | |
| 6 | 3184 | 2134 | 1050 | 33 | | | | |
| 7 | | | | | 1080 | 753 | 327 | 30 |
| 8 | 5296 | 5003 | 293 | 6 | | | | |
| 9 | 2450 | 1616 | 834 | 34 | | | | |
| 10 | | | | | | | | |
| 11 | 3956 | 3348 | 608 | 15 | | | | |
| 12 | 8630 | 8070 | 560 | 6 | | | | |
| 13 | 3450 | 2619 | 831 | 24 | | | | |
| 14 | | | | | 944 | 771 | 173 | 18 |
| 15 | 3706 | 2805 | 901 | 24 | 1258 | 953 | 305 | 24 |
| 16 | | | | | 1143 | 1039 | 104 | 9 |
| 17 | 6044 | 5524 | 520 | 9 | | | | |
| 18 | 3706 | 2949 | 757 | 20 | | | | |
| 19 | | | | | | | | |
| 20 | 3784 | 3233 | 551 | 15 | 1045 | 891 | 154 | 15 |
| 21 | 3629 | 3039 | 590 | 16 | 974 | 809 | 165 | 17 |
| 22 | 4222 | 3486 | 736 | 17 | | | | |
| 23 | 5554 | 4866 | 688 | 12 | | | | |
| 24 | 3441 | 2397 | 1044 | 30 | | | | |
| 25 | 2840 | 2579 | 261 | 9 | | | | |

^{a}Mean value as called as 2 separate mutations

### REFERENCES

All publications, patent and patent applications cited herein or filed with this application, including references filed as part of an Information Disclosure Statement are incorporated by reference in their entirety.
1. Gnirke, A. et al. Solution hybrid selection with ultra-long oligonucleotides for massively parallel targeted sequencing. Nat. Biotechnol. 27, 182-189 (2009).
2. Li, H. & Durbin, R. Fast and accurate short read alignment with Burrows-Wheeler transform. Bioinformatics 25, 1754-1760 (2009).
3. Caruccio, N. Preparation of next-generation sequencing libraries using Nextera technology: simultaneous DNA fragmentation and adaptor tagging by in vitro transposition. Methods Mol. Biol. 733, 241-255 (2011).
4. Rivas, M. A. et al. Deep resequencing of GWAS loci identifies independent rare variants associated with inflammatory bowel disease. Nat. Genet. 43, 1066-1073 (2011).
5. Lunter, G. & Goodson, M. Stampy: a statistical algorithm for sensitive and fast mapping of Illumina sequence reads. Genome Res. 21, 936-939 (2011).
6. Lange, K., Weeks, D. & Boehnke, M. Programs for Pedigree Analysis: MENDEL, FISHER, and dGENE. Genet. Epidemiol. 5, 471-472 (1988).
7. Snape, K. et al. Predisposition gene identification in common cancers by exome sequencing: insights from familial breast cancer. Breast Cancer Res Treat 134, 429-433 (2012).
8. Caruccio, N. Preparation of next-generation sequencing libraries using Nextera technology: simultaneous DNA fragmentation and adaptor tagging by in vitro transposition. Methods Mol Biol 733, 241-255 (2011).
9. Schouten, J. P. et al. Relative quantification of 40 nucleic acid sequences by multiplex ligation-dependent probe amplification. Nucleic Acids Res 30, e57 (2002).
10. Loveday, C. et al. Germline RAD51C mutations confer susceptibility to ovarian cancer. Nat Genet 44, 475-476 (2012).
11. Antoniou, A. C. & Easton, D. F. Polygenic inheritance of breast cancer: Implications for design of association studies. Genet Epidemiol 25, 190-202 (2003).

Sequence Listing

SEQ ID NO: 1 - amino acid sequence. 605 amino acid residues.
NCBI Acc. No.: NP_003611
MAGLYSLGVSVFSDQGGRKYMEDVTQIVVEPEPTAEEKPSPRRSLSQPLPPRPSPAALPGGEV
SGKGPAVAAREARDPLPDAGASPAPSRCCRRRSSVAFFAVCDGHGGREAAQFAREHLWGFIKK
QKGFTSSEPAKVCAAIRKGFLACHLAMWKKLAEWPKTMTGLPSTSGTTASVVIIRGMKMYVAH
VGDSGVVLGIQDDPKDDFVRAVEVTQDHKPELPKERERIEGLGGSVMNKSGVNRVVWKRPRLT
HNGPVRRSTVIDQIPFLAVARALGDLWSYDFFSGEFVVSPEPDTSVHTLDPQKHKYIILGSDG
LWNMIPPQDAISMCQDQEEKKYLMGEHGQSCAKMLVNRALGRWRQRMLRADNTSAIVICISPE
VDNQGNFTNEDELYLNLTDSPSYNSQETCVMTPSPCSTPPVKSLEEDPWPRVNSKDHIPALVR
SNAFSENFLEVSAEIARENVQGVVIPSKDPEPLEENCAKALTLRIHDSLNNSLPIGLVPTNST
NTVMDQKNLKMSTPGQMKAQEIERTPPTNFKRTLEESNSGPLMKKHRRNGLSRSSGAQPASLP
TTSQRKNSVKLTMRRRLRGQKKIGNPLLHQHRKTVCVC
SEQ ID NO: 2 - cDNA sequence - Start and stop codons
underlined, untranslated regions (UTRs) italicised. 6 exons,
1,818 translated bases.
NCBI Acc. No.: NM_003620
ggggaagcgcagtgcgcaggcgcaactgcctggctctgctcgctccggcgctccggcccagct
ctcgcggacaagtccagacatcgcgcgcccccccttctccgggtccgccccctcccccttctc
ggcgtcgtcgaagataaacaatagttggccggcgagcgcctagtgtgtctcccgccgccggat
tcggcgggctgcgtgggaccggcgggatcccggccagccggccatggcggggctgtactcgct
gggagtgagcgtcttctccgaccagggcgggaggaagtacatggaggacgttactcaaatcgt
tgtggagcccgaaccgacggctgaagaaaagccctcgccgcggcggtcgctgtctcagccgtt
gcctccgcggccgtcgccggccgcccttcccggcggcgaagtctcggggaaaggcccagcggt
ggcagcccgagaggctcgcgaccctctcccggacgccggggcctcgccggcacctagccgctg
ctgccgccgccgttcctccgtggcctttttcgccgtgtgcgacgggcacggcgggcgggaggc
ggcacagtttgcccgggagcacttgtggggtttcatcaagaagcagaagggtttcacctcgtc
cgagccggctaaggtttgcgctgccatccgcaaaggctttctcgcttgtcaccttgccatgtg
gaagaaactggcggaatggccaaagactatgacgggtcttcctagcacatcagggacaactgc
cagtgtggtcatcattcggggcatgaagatgtatgtagctcacgtaggtgactcaggggtggt
tcttggaattcaggatgacccgaaggatgactttgtcagagctgtggaggtgacacaggacca
taagccagaacttcccaaggaaagagaacgaatcgaaggacttggtgggagtgtaatgaacaa
gtctggggtgaatcgtgtagtttggaaacgacctcgactcactcacaatggacctgttagaag
gagcacagttattgaccagattccttttctggcagtagcaagagcacttggtgatttgtggag
ctatgatttcttcagtggtgaatttgtggtgtcacctgaaccagacacaagtgtccacactct
tgaccctcagaagcacaagtatattatattggggagtgatggactttggaatatgattccacc
acaagatgccatctcaatgtgccaggaccaagaggagaaaaaatacctgatgggtgagcatgg
acaatcttgtgccaaaatgcttgtgaatcgagcattgggccgctggaggcagcgtatgctccg
agcagataacactagtgccatagtaatctgcatctctccagaagtggacaatcagggaaactt
taccaatgaagatgagttatacctgaacctgactgacagcccttcctataatagtcaagaaac
ctgtgtgatgactccttccccatgttctacaccaccagtcaagtcactggaggaggatccatg
gccaagggtgaattctaaggaccatatacctgccctggttcgtagcaatgccttctcagagaa
ttttttagaggtttcagctgagatagctcgagagaatgtccaaggtgtagtcataccctcaaa
agatccagaaccacttgaagaaaattgcgctaaagccctgactttaaggatacatgattcttt
gaataatagccttccaattggccttgtgcctactaattcaacaaacactgtcatggaccaaaa
aaatttgaagatgtcaactcctggccaaatgaaagcccaagaaattgaaagaacccctccaac
aaactttaaaaggacattagaagagtccaattctggccccctgatgaagaagcatagacgaaa
tggcttaagtcgaagtagtggtgctcagcctgcaagtctccccacaacctcacagcgaaagaa
ctctgttaaactcaccatgcgacgcagacttaggggccagaagaaaattggaaatcctttact
tcatcaacacaggaaaactgtttgtgtttgctgaaatgcatctgggaaatgaggtttttccaa
acttaggatataagagggctttttaaatttggtgccgatgttgaactttttttaaggggagaa
aattaaaagaaatatacagtttgactttttggaattcagcagttttatcctggccttgtactt
gcttgtattgtaaatgtggattttgtagatgttagggtataagttgctgtaaaatttgtgtaa
atttgtatccacacaaattcagtctctgaatacacagtattcagagtctctgatacacagtaa
ttgtgacaatagggctaaatgtttaaagaaatcaaaagaatctattagattttagaaaaacat
ttaaactttttaaaatacttattaaaaaatttgtataagccacttgtcttgaaaactgtgcaa
ctttttaaagtaaattattaagcagactggaaaagtgatgtattttcatagtgacctgtgttt
cacttaatgtttcttagagccaagtgtcttttaaacattattttttatttctgatttcataat
tcagaactaaatttttcatagaagtgttgagccatgctacagttagtcttgtcccaattaaaa
tactatgcagtatctcttacatcagtagcattttttctaaaccttagtcatcagatatgctta
ctaaatcttcagcatagaaggaagtgtgtttgcctaaaacaatctaaaacaattcccttcttt
ttcatcccagaccaatggcattattaggtcttaaagtagttactcccttctcgtgtttgctta
aaatatgtgaagttttccttgctatttcaataacagatggtgctgctaattcccaacatttct
taaattattttatatcatacagttttcattgattatatgggtatatattcatctaataaatca
gtgaactgttcctcatgttgctgaatttgtagttgttggtttattttaatggtatgtacaagt
tgagtatcccttatccaaaatgcttgggaccagaagtgtttcagattttttaaaattttggaa
tatttgctttatactgagcttttgagtgttcccaatctgaaattcaaaatgctctaatgagca
tttcctttgagcatcatgcctgctctgaaaaagtttctgattctggagcattttggattttgg
attttcagattagggatgcttaacctggattaacattctgttgtgccatgatcatgctttaca
gtgagtgtattttatttatttattattttgtttgtttgtttgagatggagtctcactctgtca
tccaggctagagtgcagtggcgtgatctcggctgactgcaacctctgcctcccgggttcaagt
gattctcctgcctcaatctctctccccagaagctgggattacaggtgtgtgccaccacacccg
gctaatttttttttttttttttgagatggagtctagctctgtcatccaggctggagtgcagtg
gtgtgatctcggctccctgcaacctctgccttctgggttcctgcgattctcctgcctcagcct
cctgagtagctgagattacaggcacgcgccactgtgcccagccaatttttgtatttttagtag
agatggggtttcacatgtcagtcatgctggtcttgatctcctgacctcgtgatccacccgcct
cgacctcccaaagtactgggattacaggcgtgagccaccgcatccggcctgagttttatgctt
tcaatgtatttcttacatttcagttcaagtgattttcatgtctcagcctcctgagtagctgga
actacaggtgcgtgccaccatgcctggctaagttttgtatttttagtagagatgggttttcat
catgttggccaagatggtcttgatctcttgacctcatgatccaccagcctaggcctcccaaag
tgctgggattacaggtgtgagccaccgtgcccagccaactatgccattatttaaccatgtcca
cacattctggttattttcaatattttgcagaagataattcttgatcggtgtgtcttatgccac
aaggattaaaatatgtattcattgctacaaaacaatatctcgaaatttagcagtttaaaacaa
caaatattatctccagtttctgagcctcagaaatctgagagtggtttagctgggtgatagtct
cgtggttttggtcaagctaccaaccagggctacaatctttcgaaggtgtcattggggctagaa
gatctgcttcccgcaagactcacagctgttggcaggagacctcagtttgttgccacatgttcc
cctccagagggcctctcacaacatggcagttatttgtccccagagcaagcaacaccggagggc
aaggaagaagccatgatgttttttgtaacctagcctctgaaagtgtcataccaattctgtatt
ttgttggtcacacagaccaagtcaactacaacgtgggagactcctacacaaggcatgaattct
aggaggtgggcatttttaagtgtcatctggaaggaggctgtcacaacctggaagttaaaagca
ttgatattctgaaatacagcgtgtataacattgttttagtagggtgtgcaatagttatgtttt
ggtaatagcattaatgaacaatgttattttcatcttccagacatctggaagattgctctagtg
gagtaaaacatcttaatgtattttgtccctaaataaactatctcactaacaaaaaaaaaaaaa
aa

1. A method for determining whether an individual has an increased susceptibility or predisposition to cancer, the method comprising determining in a sample obtained from the individual the presence of a mutation in the PPM1D gene, or a polypeptide encoded by the PPM1D gene wherein the presence of said mutation is indicative of the increased risk of cancer.

2. The method of claim 1, wherein the cancer is breast or ovarian cancer.

3. The method of claim 1, wherein the mutation results in increased phosphatase activity of the polypeptide expressed from the PPM1D gene.

4. The method of claim 1, wherein the mutation is a truncating mutation.

5. The method of claim 1, wherein the mutation is in exon 6 of the PPM1D gene.

6. The method of claim 5, wherein the mutation is between positions 1,493 and 4,778 of SEQ ID NO: 2 (inclusive).

7. The method of claim 1, wherein the mutation is between positions 1,493 and 1,927 of SEQ ID NO: 2 (inclusive).

8. The method of claim 1, wherein the mutation is set out in Table 1.

9. The method of claim 1, wherein the step of determining the presence of a mutation in the PPM1D gene uses direct sequencing, hybridisation to a probe, restriction fragment length polymorphism (RFLP) analysis, single-stranded conformation polymorphism (SSCP), heteroduplex analysis, PCR amplification of specific alleles, amplification of DNA target by PCR followed by a sequencing assay, allelic discrimination during PCR, Genetic Bit Analysis, pyrosequencing, oligonucleotide ligation assay, or analysis of melting curves.

10. The method claim 1, wherein the DNA sequence of the PPM1D gene or the RNA sequence or cDNA sequence of a PPM1D gene product is determined.

11. The method of claim 1, wherein determining the presence of a mutation in the PPM1D gene comprises sequencing the PPM1D gene in the sample, or a portion thereof known to contain a mutation, to determine whether the mutation is present in the PPM1D gene in the sample.

12. The method of claim 10, wherein sequencing is performed using a next generation sequencing (NGS) methodology.

13. The method of claim 10, wherein sequencing is performed using Illumina sequencing, 454 pyrosequencing, Heliscope single molecule sequencing, single molecule real time (SMRT) sequencing, Ion semiconductor sequencing, Polony sequencing, SOLiD sequencing or DNA nanoball sequencing technologies.

14. The method of claim 10, wherein PPM1D is sequenced as part of a whole genome sequencing, exome sequencing or disease-associated gene sequencing project.

15. The method of claim 1, wherein determining the presence of a mutation comprises contacting nucleic acid in the sample with a sequence specific probe capable of binding to a PPM1D gene sequence comprising one or more mutations under hybridising conditions and the method comprising contacting the probe and the test sample under hybridising conditions and observing whether hybridisation takes place.

16. The method of claim 1, wherein determining the presence of a mutation in the PPM1D gene comprises digesting a sample comprising the PPM1D gene with one or more restriction enzymes to cut the nucleic acid and produce a restriction pattern for comparison with patterns obtained with a normal PPM1D gene or a mutated form thereof.

17. The method of claim 1, wherein determining the presence of a mutation comprises contacting a sample containing PPM1D gene, or a portion thereof, with one or more sequence specific primers that are capable of priming the amplification of the nucleic acid if a normal or mutated form of the PPM1D gene is present in the sample.

18. The method of claim 9 which comprises the initial step of amplifying the PPM1D nucleic acid present in the sample.

19. The method of claim 1, wherein determining the presence of a mutation comprises contacting a sample with a specific binding partner capable of specifically binding to normal or mutated PPM1D polypeptide.

20. The method of claim 19, wherein the specific binding member is an antibody.

21. The method of claim 1, wherein the step of determining the presence of a mutation uses a microarray.

22. The method of claim 21, wherein the microarray is a spotted microarray, a lithographic microarray or a bead-based microarray.

23. The method of claim 21, wherein the microarray comprises a plurality of nucleic acid probes or a plurality of antibodies.

24. A method which comprises having determined whether an individual has an increased susceptibility to cancer according to the method of claim 1, one or more of the further step of:
(a) correlating the presence of said mutations to a susceptibility to breast cancer or ovarian cancer; and/or
(b) saving data representing the result of the test on a recordable media; and/or
(c) transmitting the data representing the result of the test to a recipient.

25. A kit for detecting mutations in the PPM1D gene associated with a susceptibility to cancer according to claim 1, the kit comprising:
(a) one or more sequence specific probes as set out in claim 15; and/or
(b) one or more sequence specific primers for amplifying a portion of the PPM1D nucleic acid sequence; and/or
(c) one or more specific binding partners capable of specifically binding to normal or mutated PPM1D polypeptide as set out in claim 19; and/or
(d) a microarray.

26. A method of treating cancer, the method comprising determining whether an individual has an increased predisposition to cancer according to the method of claim 1 and, where the individual has a mutation in the PPM1D gene, treating the individual with the PPM1D inhibitor.

27. The method of treating cancer according to claim 26, wherein the cancer is breast cancer or ovarian cancer.
